(19) United States
(12) Patent Application Publication
(10) Pub. No.: US 2012/0226146 A1
(43) Pub. Date: Sep. 6, 2012

(54) DEPLOYABLE MARKER AND METHOD FOR DEPLOYMENT

(76) Inventor: Jeremy Schwartz, New York, NY (US)

(21) Appl. No.: 13/409,859

(22) Filed: Mar. 1, 2012

Related U.S. Application Data
(60) Provisional application No. 61/447,888, filed on Mar. 1, 2011.

(51) Int. Cl. A61B 5/05; G02B 7/182

(52) U.S. Cl. 600/424; 359/871

(57) ABSTRACT

A marker for marking the location of an internal area in the human body comprising a cap unit, and at least one attachment unit affixed to the cap unit, the at least one attachment unit capable of piercing a surface of a region in response to a vertical force applied to the cap unit.

### CROSS-REFERENCE TO RELATED APPLICATIONS

The present application claims the benefit of U.S. Provisional Application Ser. No. 61/447,888 filed 1 Mar. 2011, which is incorporated herein by reference in its entirety.

### FIELD OF THE INVENTION

This disclosure relates to deployable endoscopic markers and methods for deploying endoscopic markers.

### BACKGROUND

In the course of gastrointestinal (GI) endoscopy, an endoscopist frequently encounters the need to mark a specific location and/or finding either to aid in its subsequent identification or to define its borders. Sometimes this is done in order for the endoscopist to be able to relocate a specific site at a later point during that particular procedure or during a future procedure. Marking is also done such that another endoscopist or a surgeon can identify a particular finding or location at a later date. Finally, there are times during a procedure that it is necessary to identify and subsequently mark the borders of a particular finding or lesion in order to aid in its accurate and complete removal. Current methods of marking, which include tattooing, creating cautery marks, and clipping, is limited by risks of procedural complications, lack of effectiveness, high cost, and types of findings which are not amenable to the aforementioned methods of marking.

There are three current methods used for marking in gastrointestinal endoscopy. The first method involves the submucosal injection of a dye or ink and is known as tattooing. This method involves the placement of a catheter with an extendible needle-tip through the endoscope. A syringe with ink is attached to the proximal end of this needle-tipped catheter. Within the desired area to be marked, the needle-tip is extended and maneuvered through the mucosal layer and into the submucosal layer. The ink is then injected submucosally to create a tattoo marking which can be seen from both the mucosal and serosal surfaces. Within some areas of the gastrointestinal tract, multiple injections are often required in order to achieve adequate visualization from a serosal viewpoint, particularly within the colorectum. These tattoo marks are permanent and can be helpful to a surgeon in identifying the general region that a particular abnormality (i.e. a colon cancer) is in as well as to the endoscopist trying to re-identify an abnormality (i.e. the site of a prior polyp removal).

In spite of its widespread use, tattooing has multiple drawbacks. Technical control of the injection needle, even in experienced and expert hands can often be difficult, leading to frequent failed injections or deep injections with tattoo ink entering the peritoneal cavity which can lead to serious consequences. There is also a risk of perforation of the wall of the GI tract and/or bleeding due to technical failures in controlling the injection needle. Furthermore, the tattoo marking is often relatively large and as such tattooing often marks a general region as opposed to a specific site, thereby sometimes making definitive and precise identification of a specific finding difficult even under the best of circumstances. The tattoo marking can also be hard to find due to its faintness or its confusion with other markings (i.e. the hepatic and splenic flexures within the colon). Lastly, reactions within the bowel wall to ink (i.e. India ink) or other tattoo agents employed include edema, necrosis, and neutrophilic infiltration and are not uncommon.

Another method in the current art for marking employs cauterization to mark the outer borders of a lesion about to undergo endoscopic resection (i.e. via snare electrocautery, endoscopic mucosal resection, or endoscopic submucosal dissection) such that the endoscopist can have clear demarcation of these borders in order to achieve a successful resection of the abnormal tissue without unnecessarily removing normal tissue. This marking is often achieved using a needle-knife device and applying cauterization in a “dotlike” fashion around the perimeter of the lesion to be removed. This method is useful but it too has its shortfalls. Use of cautery within the gastrointestinal tract can always lead to thermal injury of the bowel wall, potentially leading to a full-thickness injury of the bowel wall with subsequent bowel wall perforation. This marking method is not always optimal due to the often low degree of contrast between the cauterized areas (whitish) and the adjacent mucosa (pinkish). In addition, the ability to distinguish between cauterized and background mucosa often diminishes during the course of the procedure due to tissue edema as well as the application of cautery to the lesion itself.

A third method for marking employs a device called an endoscopic clip. The primary functions of these clips are to close defects in the wall of the GI tract and to control bleeding. They also, however, have an FDA-approved indication for endoscopic marking. These clips are deployed by opening their “teeth” and then “biting” the mucosa. They are then closed and remain attached to the mucosa by pinching it. They are stainless steel and show up on radiographic studies. They also stick out from the wall of the GI tract and can therefore be seen (silver in color) during endoscopy as well.

Endoscopic clips have several disadvantages. They are stainless steel which is ferromagnetic—precluding MRI studies while in place. In addition, their color makes them difficult to identify, and they are difficult to deploy and have frequent failures. Additionally, endoscopic clips attach to the mucosal layer of the wall of the GI tract and not its deeper layers. As such, these clips often spontaneously slough off of the wall of the GI tract within only a few days of having been placed. When it becomes necessary to remove them, there can be difficulties and occasional complications. Lastly, they are expensive. These disadvantages make the routine deployment of the multiple clips needed for endoscopic marking both impractical and cost prohibitive. Three companies which make endoscopic clips are Wilson-Cook Medical (tri-clip endoscopic clip with three teeth), Olympus America Inc. (bi-clip endoscopic clip with two teeth) and Boston Scientific Corp. (bi-clip endoscopic clip which can be repeatedly opened and closed).

Therefore, there is a need for a novel marking system for use during GI endoscopy that is effective, safe, cost-effective, and not cumbersome to employ and which improves localization and identification of specific findings while avoiding the risks to the patient which are associated with the current art.

### SUMMARY OF THE INVENTION

What is proposed is a tubular element for passage through the therapeutic channel of a GI endoscope. At the proximal end of the tubular element is a control handle that controls actuators which function via the tubular element to effect placement of a deployable marker onto and/or into the wall of the GI tract in order to mark a particular area or finding. In different embodiments of this device the number of deployable markers per device may vary as would the composition of the marker itself (i.e. stainless steel, ceramic, titanium, nitinol, rubber, polymer, plastic, biodegradables, and/or digestibles). The color of the marker will vary by embodiment; however, in one embodiment, the color will always contrast significantly with the color of the surrounding background mucosa. In another embodiment, the markers will also show up on radiographic studies. This may be accomplished by constructing a hybrid marker molded with fine titanium particles suspended within a first material (i.e. polymer). In different embodiments of this device, the means of attachment as well as the depth of penetration of the marker to the GI tract wall may vary. The marker may penetrate through the entire wall of the GI tract layers and anchor. A deeper penetration of the marker into the GI tract wall may be associated with more permanent endoscopic marking. Deployment of the marker to anchor into the serosal surface, in addition to allowing for a more permanent marking, may be useful for serosal side identifications as well as for more permanent marking indications. Other embodiments would deploy a shallower marker anchoring to either the mucosa, submucosa, or muscularis propria. Such embodiments would be useful when marking was needed solely for mucosal side identification(s) as well as for less permanent marking indications. An additional solution for a less-permanent endoscopic marker would involve constructing the marker or portions of the marker out of biodegradable and/or digestible materials. This endoscopic marker system, as described, is an elegant solution to the limitations and drawbacks of the current art in endoscopic marking technology.

### BRIEF DESCRIPTION OF THE DRAWINGS

FIGS. 1A-1F are side views of various embodiments of an endoscopic marker according to one embodiment.

FIG. 2 is a top view of a delivery catheter for an endoscopic marker according to one embodiment.

FIG. 3 is a cross-sectional view of a polyp seen on insertion of a colonoscope, with a cross sectional view of the GI tract, marked with either one or a plurality of markers according to one embodiment so that the polyp can be later relocated.

FIG. 4 is a top view of a lesion for endoscopic mucosal resection, saline snare resection or endoscopic submucosal dissection, marked with a plurality of endoscopic markers according to one embodiment so that the lesion can then be removed by the endoscopist with confidence that the lesion's margins have been clearly delineated.

FIG. 5 is a top view of a submucosal nodule or other finding marked with a plurality of endoscopic markers according to one embodiment so that the finding can be rechecked at a later time and possibly examined in more detail with endoscopic ultrasound (EUS). These markers can also help identify this lesion should it need to be removed either surgically or endoscopically.

FIG. 6 is a side view of a section of the GI tract with a neoplasm marked with endoscopic markers according to one embodiment for later rechecking and/or removal.

FIGS. 7A-7E depict one embodiment of a marker.

FIG. 8A is a perspective view of one embodiment of a marker.

FIG. 8B depicts a bottom view of one embodiment of a marker.

FIG. 9 depicts a side view of one embodiment of a spring included in a marker.

FIG. 10 depicts a perspective view of one embodiment of a marker.

FIG. 11 depicts one embodiment of a marker.

FIG. 12A depicts a side view of one embodiment of a marker.

FIG. 12B depicts one embodiment of a delivery unit for a marker.

FIG. 13A depicts the bottom view of one embodiment of a marker.

FIG. 13B depicts a top view of one embodiment of a marker.

FIG. 13C depicts one embodiment of a delivery device for the marker.

FIG. 14A depicts two marking units.

FIG. 14B depicts a marker connected to a delivery unit.

FIG. 15A depicts one embodiment of a marker unit.

FIG. 15B depicts one embodiment of a delivery unit for a marker.

FIG. 16A depicts one embodiment of a marker.

FIG. 16B depicts one embodiment of a marker.

FIG. 17A depicts one embodiment of a maker.

FIG. 17B depicts one embodiment of a delivery unit for a marker.

FIG. 17C depicts a marker engaging a region.

FIG. 17D depicts a marker engaging in to a region.

FIG. 18A depicts one embodiment of a marker.

FIG. 18B depicts one embodiment of a delivery unit for a marker.

FIG. 19A is one embodiment of a marker.

FIG. 19B depicts one embodiment of a deliver unit for a marker.

FIG. 19C depicts an illustrative example of a marker engaged with a region.

FIG. 20A depicts a top view of one embodiment of a marker.

FIG. 20B depicts one embodiment of a delivery unit for a marker.

FIG. 20C depicts an illustrative example of a marker engaged with a region.

FIG. 21A depicts a side view of one embodiment of a marker.

FIG. 21B depicts one embodiment of a delivery unit for a marker.

FIG. 21C depicts one embodiment of a delivery unit for a marker

FIG. 21D depicts a marker embedded in the region.

FIG. 22A depicts a side view of one embodiment of a marker.

FIG. 22B depicts one embodiment of a marker.

FIG. 22C depicts a side view of one embodiment of a marker.

FIG. 22D depicts the side view of a marker.

FIG. 22E depicts a bottom view of a marker.

FIG. 22F depicts a side view of a marker.

FIG. 22G depicts a side view of a marker.

FIG. 22H depicts a side view of a marker.

FIG. 23A depicts a top view of one embodiment of a marker.

FIG. 23B depicts one embodiment of a delivery unit for a marker.

FIG. 24A depicts a cut away side view of a marker.

FIG. 24B depicts one embodiment of a delivery unit for a marker.

FIG. 25A depicts one embodiment of a marker.

FIG. 25B depicts one embodiment of a marker with a locking unit.

FIG. 25C depicts a delivery unit for a marker.

FIG. 26A depicts a side view of a marker.

FIG. 26B depicts a side view of a marker.

FIG. 26C depicts a side view of a connection between an insertion unit and a plurality of wires.

FIG. 27A depicts a top view of a material adhered to a region using a plurality of markers.

FIG. 27B depicts a top view of a plurality of markers surrounding an opening in the wall of the GI tract.

FIG. 28A depicts a top view of one embodiment of a mirror device.

FIG. 28B depicts a top view of one embodiment of a mirror device.

FIG. 29A depicts a side view of a marker.

FIGS. 29B-29D depict a delivery unit for a marker.

FIG. 30A depicts a top view of a marker.

FIG. 30B depicts a side view of a marker.

FIGS. 30C-30E depict a delivery unit for a marker.

### DETAILED DESCRIPTION OF THE INVENTION

The marker may be of any shape, including round, square and triangular. The shape of the marker may also be that of its legs or other attachment means. Preferably, the marker will be round.

The marker may be of any convenient diameter. Preferably, the marker will be about 2-4 mm in diameter. The marker may be made of any suitable material. Preferably, the marker may be made of a material which is not irritating to the mucosa or any other layer of the wall of the GI tract. More preferably, the marker may be made of materials including: stainless steel, titanium, nitinol, rubber, polymer, ceramic, plastic, biodegradables, digestibles, or any combination of these materials.

The marker may be of any suitable color or colors. Preferably, the marker will be of a color which contrasts with the color of the surrounding GI tract wall. In one embodiment, the marker will be black. This may be achieved by either constructing the marker of a black material or by coloring the marker black. If coloring is used, the coloring agent will preferably be non-toxic.

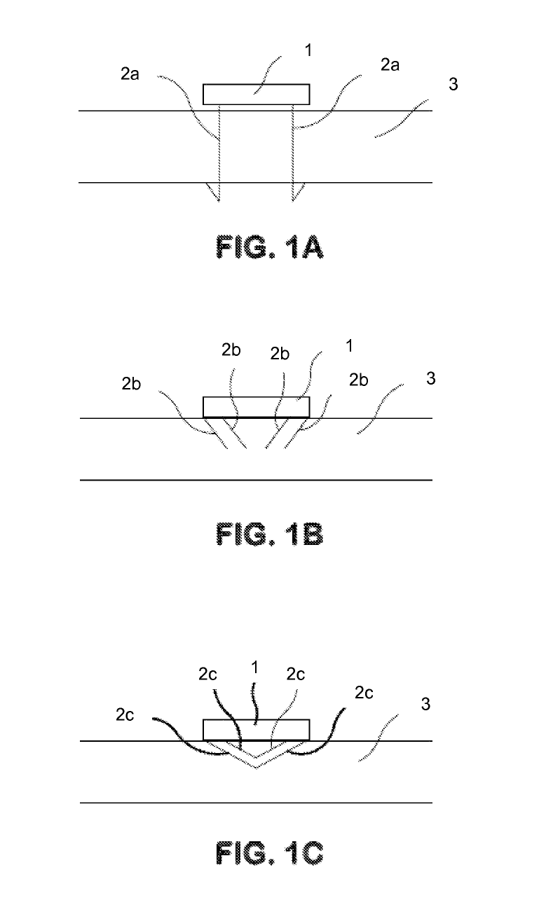

The marker will be attached to the mucosa and/or deeper wall layers of the GI tract via an attachment means. The attachment means may either be permanent or reversible depending on the specific procedure. Various attachment means are shown in FIGS. 1A-1F. Preferably, the attachment means will be reversible, i.e. the marker will be detachable. In one embodiment, the markers are permanently attached. In another embodiment, a part of or all of the marker is made from a biodegradable and/or digestible material which dissolves over time in order to render the marker non-permanent and/or to dissolve any sharp features of the marker following deployment. The amount of time the material will remain attached is adjustable and depends on the amount and/or type of dissolvable material in the marker. In another embodiment, the markers can be removed, i.e., detached. As an illustrative example, a marker may be removed endoscopically by using a device similar to an endoscopic staple remover. Consistent with this embodiment, the endoscopic staple remover is inserted endoscopically and the markers are detached and removed. It is understood that the remover could take other forms such as modified biopsy forceps. Additionally, the marker itself can be designed to collapse, to disassemble, or to dissolve.

FIG. 1A shows one embodiment of proposed marker 1. The marker is attached via a plurality of legs 2a, 2a to the wall of the GI tract 3. Note that in this configuration, the legs 2a, 2a pass entirely through all of the layers of the wall of the GI tract 3, although it is understood that a marker of this configuration whose legs do not pass entirely through all the walls of the GI tract is also possible . Also note that while FIG. 1A shows two legs 2a, 2a, it is understood that one or a plurality of legs could be used.

As noted above, in one embodiment, the marker is detachable from the wall of the GI tract. In an alternative embodiment, however, the marker is not detachable. As noted above, this embodiment would be useful for serosal side identifications as well as for more permanent marking identifications. FIG. 1A, where the legs 2a, 2a penetrate through all of the layers of the wall of the GI tract 3 and anchor into the serosal surface, could be useful in this embodiment. Also, in yet another embodiment, the portion of the legs 2a, 2a which protrude through the GI wall could be configured in a design pattern, such as a square, a triangle or a star. This would aid in serosal side identifications. In an alternative, the portion of the legs 2a, 2a which protrude through the GI wall could be of a color to make them stand out.

FIG. 1B shows an alternative embodiment of the proposed marker 1. Here, the marker will have a plurality of legs 2b, 2b, 2b, 2b which will project into but preferably not through the GI wall 3. In this embodiment, the legs 2b, 2b, 2b, 2b; will project inward.

Although this embodiment shows four legs; 2b, 2b, 2b, 2b; the marker is not limited to four legs and may include additional or fewer legs. Any number of legs will do, so long as the leg or legs secure the marker 1 to the GI wall. Moreover, this statement will be true for all of the embodiments.

FIG. 1C shows another embodiment. Here, like the embodiment of FIG. 1B, the legs 2c, 2c, 2c, 2c projecting inward. Here, however, the ends of the legs 2c, 2c, 2c, 2c would meet or be in close proximity to at least one other leg end. Again, as above, although four legs 2c, 2c, 2c, 2c are shown, the marker is not limited to only four legs and may have more than four legs or fewer than four legs. Also, as shown, the legs 2c, 2c, 2c, 2c will preferably project into but not through the GI wall 3.

FIG. 1D is yet another embodiment. In this embodiment, the marker 1 has a plurality of legs 2d, 2d, projecting from about the center of the bottom of the marker 1 in a generally outward direction and into the GI wall 3. Preferably, the legs 2d, 2d do not project through the GI wall 3.

FIG. 1E shows another embodiment of a marker. This embodiment shows a plurality of legs 2e, 2e which projects outward from the marker 1. Unlike the embodiment of FIG. 1D, however, the legs 2e, 2e of FIG. 1E do not project outwardly from the center of the marker 1. Rather, they project from a position towards the edge of the marker 1 or, as shown here, at or near the edge. Also, as shown in FIG. 1E, the legs 2e, 2e preferably do not extend all the way through the GI wall 3.

FIG. 1F is yet another embodiment. This embodiment does not have a marker of the type shown in the other figures. Rather, it is the legs 2f, 2f projecting outward which performs the marking function, such as via color, design, etc. As shown, the legs 2f, 2f preferably does not pass all the way through the GI wall 3.

The above embodiments show that the marker 1 is attached to the GI wall 3 through various combinations of legs. However, any attachment means will suffice. In one embodiment, the marker is temporarily attached. In another embodiment, the marker is permanently attached.

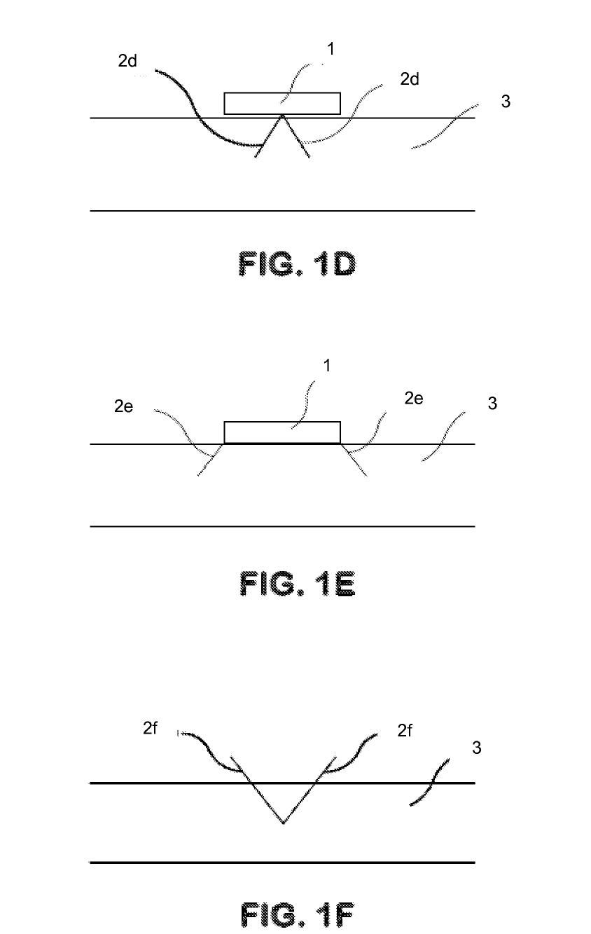

FIG. 2 shows a delivery system for the markers. The delivery system is catheter based and FIG. 2 shows a tube or catheter 4 deploying a marker 1 from its distal end. The marker 1 attaches to the GI wall to mark a finding by the endoscopist. The delivery system may preferentially include a control device which may include a gun type control, a knob, or a lever at its proximal end (not shown) to control deployment and placement of the marker 1, although any control device will work. Also, the delivery system can be loaded with one or a plurality or a packet containing a plurality of markers (not shown) and the packet can contain an optimal number of rounds, i.e., number of markers, e.g., 3, 6 or 10 markers per packet. Individual markers may be constrained (i.e. prevented from falling out prematurely) within the catheter-based delivery system until deployed by the endoscopist. This may be accomplished via looping threads or another threadlike material around each marker or by placing a membrane or membrane-like structure of material underneath each marker with the endoscopist breaking the aforementioned thread(s), threadlike material(s), membrane(s), or membrane-like material(s) via the control handle or by some other mechanism during deployment of the marker. However, any type of constraining device which acts to prevent premature marker release form the delivery system will do. The tube or catheter 4 may be reusable, disposable, or reposable.

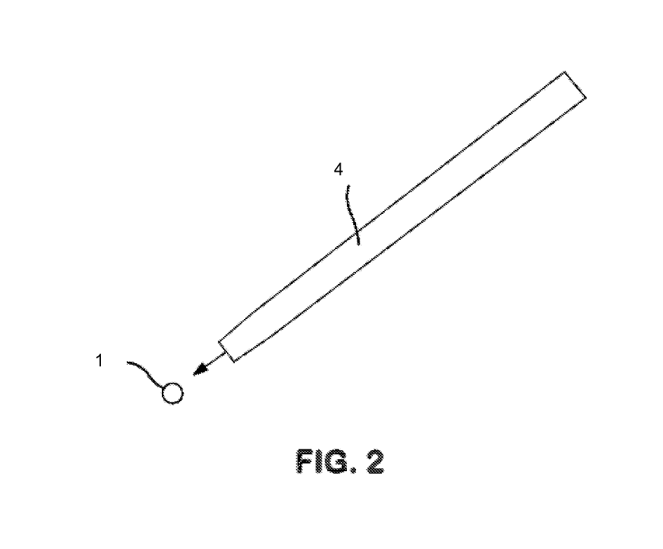

FIG. 3 is a side view of a polyp 5 protruding from the GI wall of the GI tract 3. The polyp 5 is marked with two markers 1. Here, the markers 1, are of the type shown in FIG. 1E, however, any number of markers may be used.

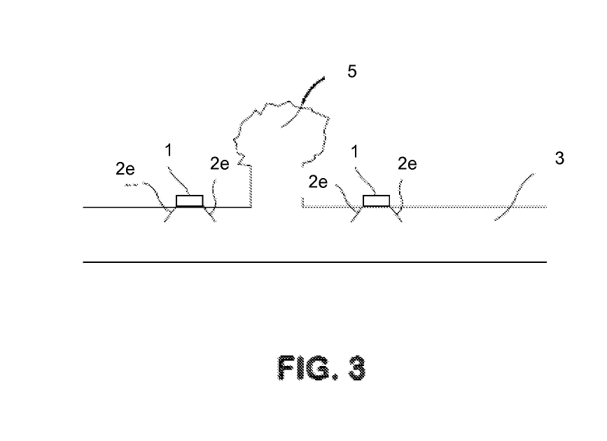

FIG. 4 shows a top view of a lesion 6 in the wall of the GI tract 3. The lesion 6 is surrounded by a plurality of markers 1. The lesion 6 can then be removed by the endoscopist with confidence that its margins have been clearly delineated.

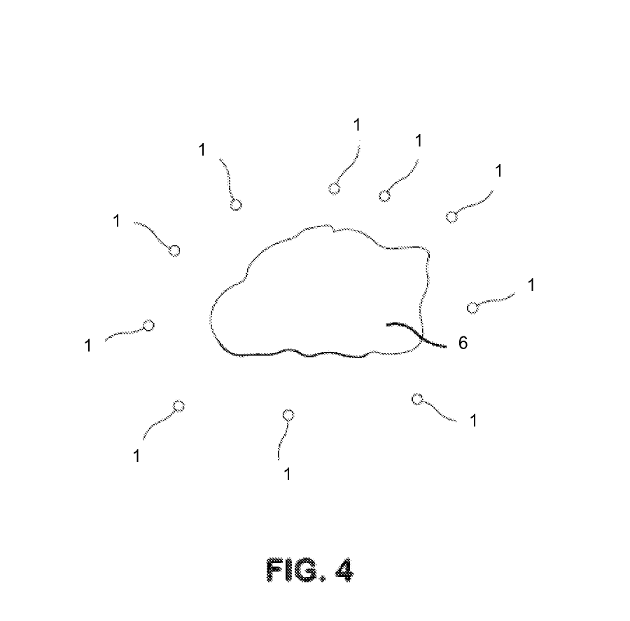

FIG. 5 shows a top view of a submucosal nodule or other finding 7 in the wall of the GI tract 3. The submucosal nodule or other finding 7 is marked with a plurality of markers 1, so that the initial endoscopist or another endoscopist can relocate the lesion at a later time for further evaluation using endoscopic ultrasound (EUS) or other means of evaluation or to possibly sample or remove the lesion. Additionally, marking such a lesion makes the lesion easier for a surgeon to identify and remove.

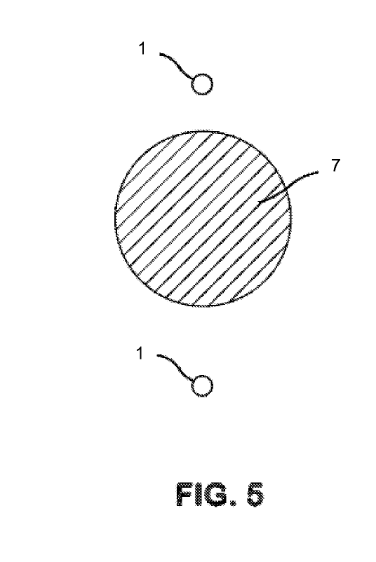

FIG. 6 is a side view of a portion of the GI tract 64 with a neoplasm 62 on or in its wall. The neoplasm 62 will be marked with a plurality of markers. As shown in FIG. 6, three markers 60 will be proximal to the neoplasm 62 and in a roughly triangular configuration around the circumference of the wall of the GI tract and three markers 66 will be distal to the neoplasm 62 and in a roughly triangular configuration around the circumference of the wall of the GI tract. This type of marking is particularly useful, for example, to mark a portion of the GI tract to be resected. It should be noted that the type, number or placement of markers to be used is not limited.

The markers are intended for use during endoscopic examination. There, an endoscopist will see a finding of interest and endoscopically mark that finding with one or more markers, using a deployment device like that shown in FIG. 2. The endoscopist can then continue the examination and then, at a later time, more easily return to the finding because it has been marked. The marking also makes it easier for another endoscopist to locate the finding.

In another embodiment the markers can be removed, i.e. detached. As an illustrative example, the marker is removed endoscopically by using a device similar to an endoscopic staple remover. Consistent with this embodiment, the remover is inserted endoscopically and the markers are detached and removed. It is understood that the remover could take other forms, such as modified biopsy forceps. Additionally, the marker itself can be designed to collapse, to disassemble, or to dissolve.

In one embodiment, the markers do not have to be removed immediately or shortly after placement. Here, an endoscopist can then examine the finding at a later time to see if there have been any changes. In an alternative embodiment, the markers are not removable.

In one embodiment, such as FIG. 1A, the legs 2a, 2a of the marker 1 would penetrate through all GI wall layers 3 and embed or anchor into the serosal surface. This embodiment would be particularly useful for serosal side identification of a finding. For example, this embodiment could be used to clearly identify a portion of the intestines which needs to be resected.

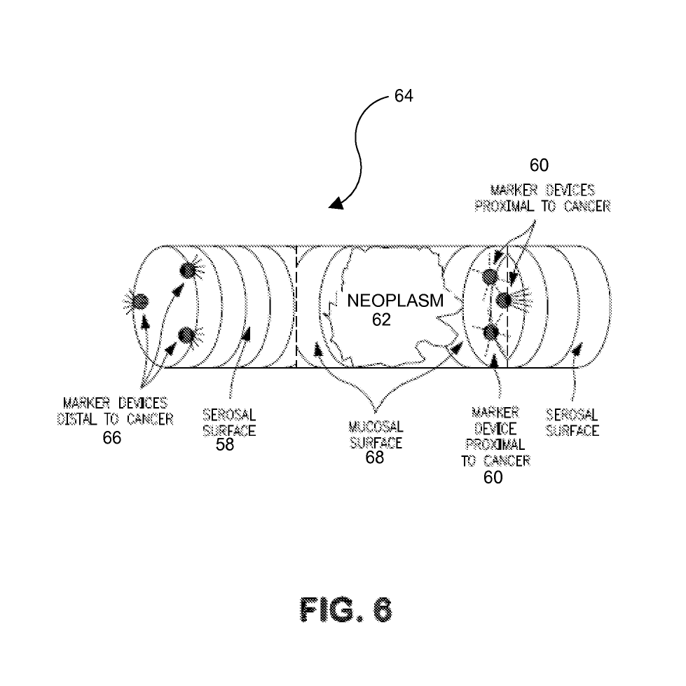

FIG. 7A depicts a side view of a marker 70. The marker 70 consists of a cap unit 72 that is coupled to at least two springs 74 and 76 which are, in turn, rotatively coupled to two metal hooks 78 and 80. In one embodiment, the cap unit 72 is color coded. In another embodiment, the cap unit 72 includes an RFID tag which includes different identifying information for each marker. In another embodiment, the cap unit 72 includes a unique letter, number or symbol on the top surface of the cap unit 72.

FIG. 7B depicts a side view of a delivery unit 81. The delivery unit 81 includes a tube 82 having an opening 83 at one end of the tube 82. A plurality of markers are stacked in the tube 82 such that the cap units 72 of a lower marker 86 that is close to the opening is in contact with the lower portion of another marker 88 that is above the lower marker. In addition, each of the metal hooks 78 and 80 are retracted into a position where the end of each hook is parallel with its respective spring 74 and 76 and the ends of each hook 78 and 80 are in contact with the inner wall of the tube 82. FIG. 7C depicts the side view of a marker being implanted onto a region 84. The lower marker 86 is pushed downward through the opening 83 towards the region 84 by a vertical transmission unit (not shown). The springs 74 and 76, which are located at a position lower than the hooks 78 and 80 and the cap unit 72 in the tube 82, indent the region 84 in contact with the springs 74 and 76. As the marker 86 is further pushed out of the tube 82, the region 84 is further indented until the hooks 78 and 80 become free of the inner walls of the tube 82 and are forced downward onto the region by the springs 74 and 76.

FIG. 7D depicts a side view of the hooks 78 and 80 engaging the region 84. Each of the springs 74 and 76 exerts a torsional force on the hooks 78 and 80 forcing the hooks 78 and 80 into the region 84. FIG. 7E depicts a side view of a marker 86 engaged with the region 84. After the delivery tube 82 is pulled away, the region 84 pushes upward against the marker 86 forcing the cap unit 72 upward. The torsional force applied on the hooks 78 and 80 by the springs 74 and 76 force the hooks 78 and 80 into the edges of the region 84 such that the edges of the region 84 are forced downward as the marker 86 is forced away from the region. Consequently, the springs 74 and 76 maintain a torsional force on the hooks 78 and 80 sufficient to prevent the edges of the region 84 from moving upward towards the cap of the marker 86.

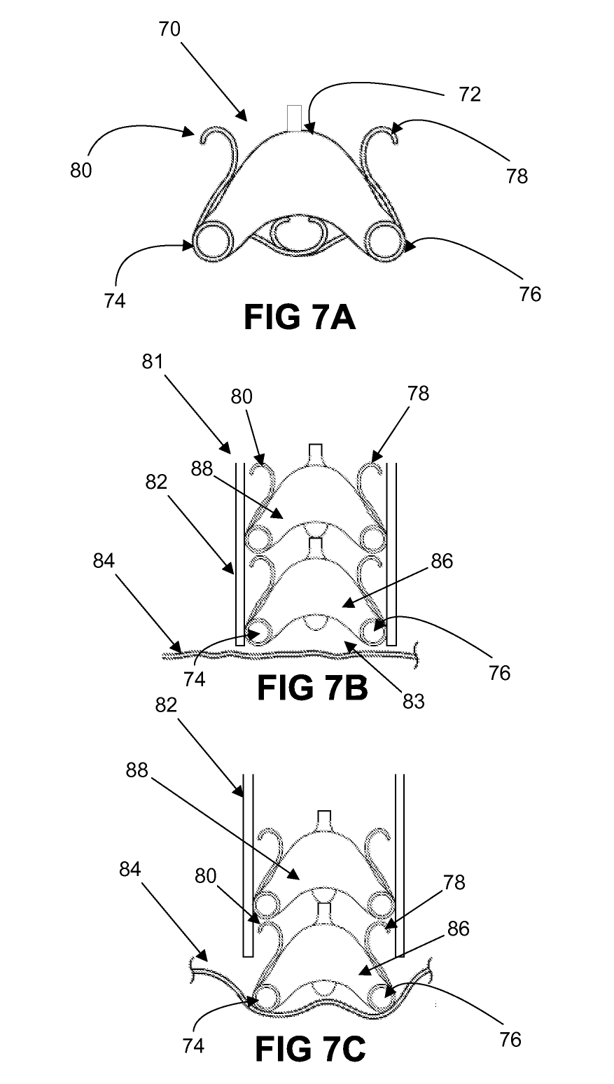

FIG. 8A is a perspective view of the marker 90. The marker 90 includes hooks 92, 94 and 96 and a cap 98. In one embodiment, the cap may include an extension 100 which engages an opening in the lower portion of another marker in the delivery tube such that the downward force applied by the vertical transmission unit is concentrated on the center portion of each marker.

FIG. 8B depicts a bottom view of the marker 90. The marker 90 includes three springs 102, 104, 106 attached to a center holding unit 108. In one embodiment, the holding unit 108 is a substantially round plate with a center portion slightly elevated above the edges of the holding unit 108. In addition, the holding unit 108 has at least three slots 110, 112 and 114. Each slot is configured to accommodate the center portion of one of the springs 102, 104 and 106. The holding unit 108 also includes a hole 116 located in the center of the holding unit 108 which accommodates the extension 100 of another marker when positioned in a delivery unit.

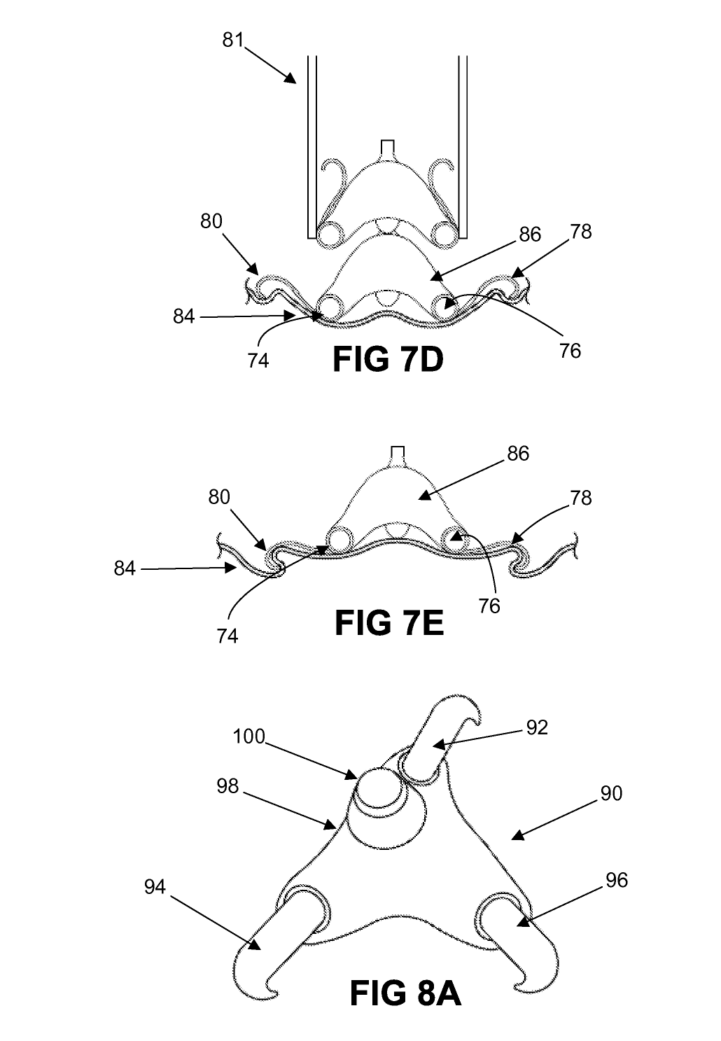

FIG. 9 depicts a side view of a spring used in a marker. The spring includes a center portion 118, an extension portion 120 that connects to a hook 122 and a locking portion 121. In one embodiment, the hook is substantially a “C” shape. However, the hook 122 may be formed into any shape capable of securing the marker to a region.

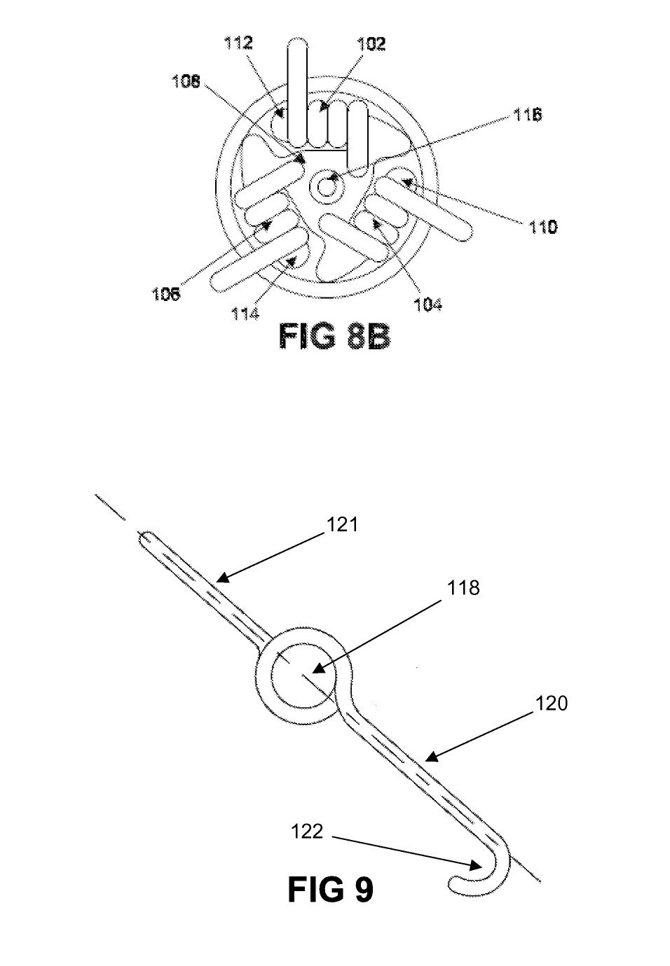

FIG. 10 depicts a perspective break away view of a marker. The marker 128 includes a cap unit 130 having an upper portion 132 and a pin 134 in the lower portion of the cap unit 130 that engages a hole 136 in a holding unit 138. The holding unit 138 includes a portion elevated above the edges of the holding unit 138 which creates a dome in the center of the holding unit 138. In addition, the holding unit 138 includes the slots 140, 142 and 144 which are configured to accommodate the center portions of the springs 146, 148 and 150. The springs 146, 148 and 150 each have an associated hook 152, 154 and 156 and associated extension portions 158, 160 and 162. The extension portions 158, 160 and 162 engage the holding unit such that each spring 146, 148 and 150 provides a torsional force to each of the hooks that act to force the hooks 158, 160 and 162 downward towards a region.

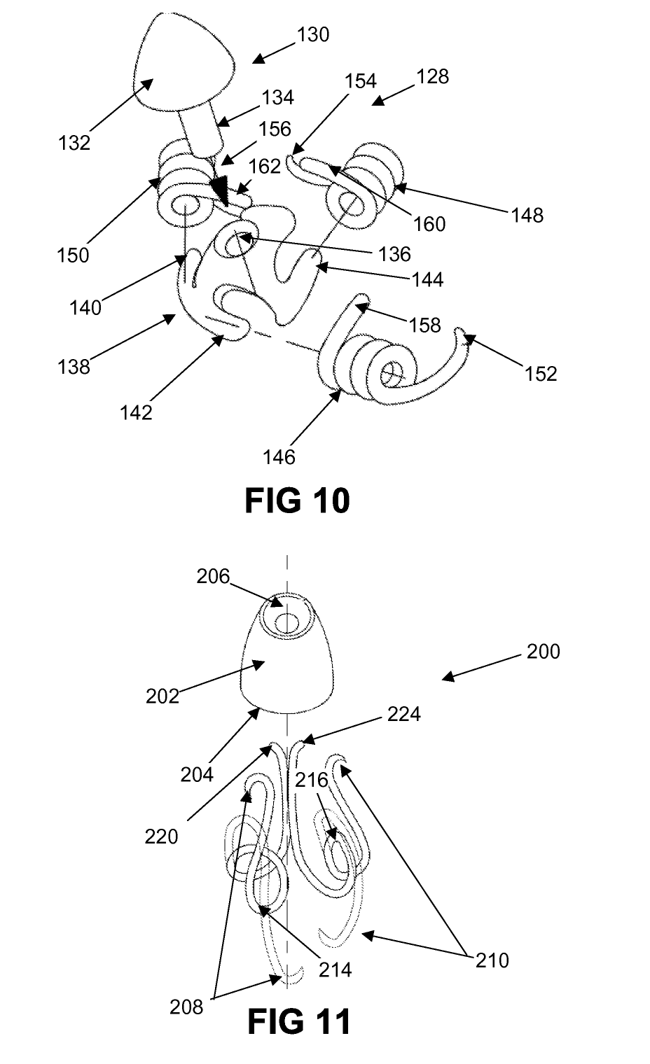

FIG. 11 depicts a perspective view of a marker 200. The marker 200 includes a cap 202 having a first opening 204 and a second opening 206 where the first opening 204 is larger than the second opening 206. The marker 200 includes at least two hooking units 208 and 210. The hooking units 208 and 210 each include a spring unit 214 and 216 and a securing unit 220 and 224. The securing units 220 and 224 include an extension portion and an end portion that are angled outward. The securing units 220 and 224 are positioned through the first opening 204 such that the angled ends of the securing units 220 and 224 protrude through the second opening 206 such that the angled portions of the ends extend over the exterior of the second opening.

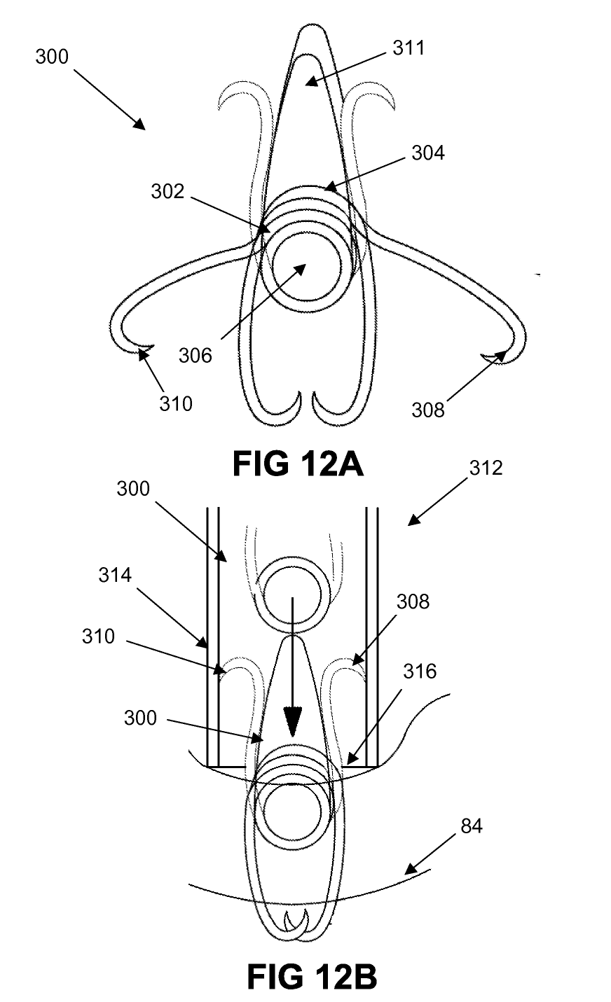

FIG. 12A depicts a side view of a marker 300. The marker 300 includes at least two springs 302 and 304 that are wrapped around a bar 306. Each spring 302 and 304 includes at least one hooking portion 308 and 310 attached to the spring. The at least two springs 302 and 304 are affixed to the bar 306 such that each spring 302 and 304 applies a torsional force downward on the hooking portions 308 and 310. In another embodiment, the marker 300 has a cap 311. In another embodiment, the cap 311 is colored to provide an indication of where the marker 300 is positioned. In another embodiment, the cap 311 may include an RFID device which is used to identify each marker. In another embodiment, the cap 311 includes a unique letter, number or symbol on the top surface of the cap.

FIG. 12B depicts a delivery unit 312 for the markers 300. The delivery unit 312 includes a tube 314 that has an opening 316 at one end. Each marker 300 is stacked in the tube 314 and is forced towards the opening 316 by a vertical transmission device (not shown). A plurality of markers are stacked in the delivery tube such that the top portion of a first marker 300 that is closer to the opening 316 is in contact with the lower portion of a second marker located above the first marker. The vertical transmission unit exerts a downward force concentrated on the center of the cap unit 311 of the each marker causing the plurality of markers to move downward in the tube 314. In addition, the hooking portions 308 and 310 of the marker are rotated around the spring such that the ends of the hooks are in contact with the sidewalls of the tube 314. As the markers 300 are forced down the tube 314, the hooking portions 308 and 310 are released at a position just above a region 84. The torsional force of the springs force the hooking portions 308 and 310 downward towards the region 84 as the marker is forced out of the tube by the vertical transmission unit.

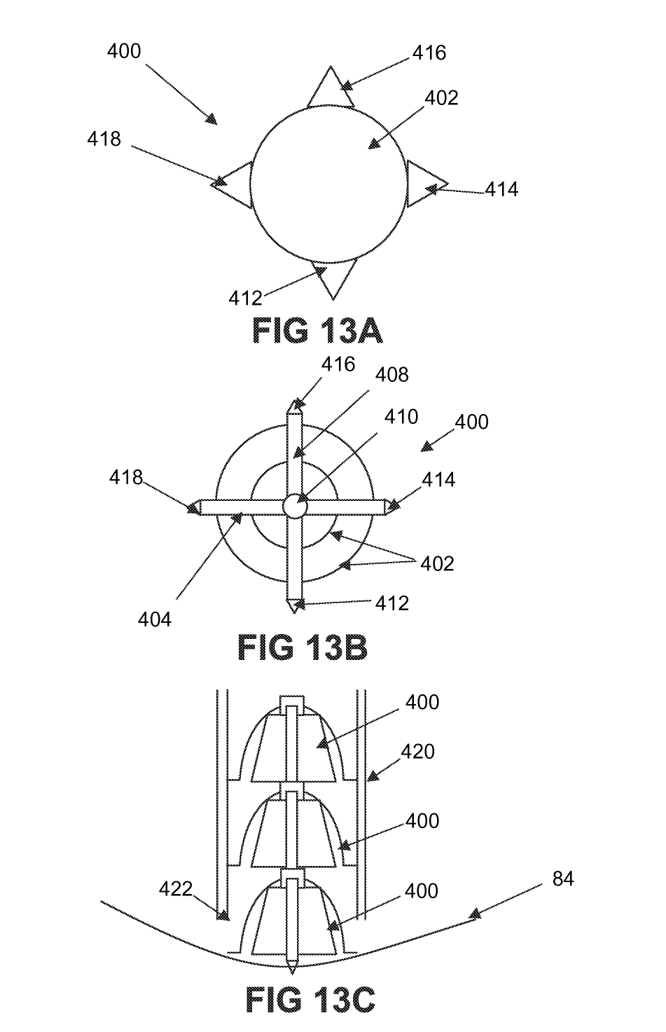

FIG. 13A depicts a bottom view of a marker 400 and FIG. 13B depicts a top view of a marker 400. The marker 400 includes a center cap 402 and two hooking units 404 and 408 having a first end and a second end. The hooking units 404 and 408 are formed over the sides of the center cap 402 and are secured to the center cap 402 by a pin 410. Each end of the hooking units 404 and 408 includes a tab 412, 414, 416 and 418 which extends outward from the hooking units 404 and 408 at approximately a right angle. The hooking units 404 and 408 are curved such that they extend outward from the cap.

FIG. 13C depicts a delivery unit for the marker 400. As the figure depicts, each marker 400 is stacked in a delivery tube 420 which includes a vertical transmission device (not shown) and an opening 422. A plurality of markers are stacked in the delivery tube 420 such that the top portion of a first marker 400 that is closer to the opening 422 is in contact with the lower portion of a second marker located above the first marker. The vertical transmission unit exerts a downward force concentrated on the center of the pin 410 of each marker causing the plurality of markers to move downward in the tube 420. In addition, the tabs 412, 414, 416 and 418 of the hooking portions 404 and 408 of the marker are forced toward the center of the delivery tube 420 by the sidewalls of the tube 420. During deployment, the tube 420 forces the region 84 downward as the marker 400 is moved toward the region 84. As the marker 400 exits the tube 420 through the opening 422, the tabs 412, 414, 416 and 418 are forced outward to engage the indented region 84. The spring force of the hooking units 404 and 408 forces the tab unit into the region 84. As the delivery unit is pulled away from the region 84, the region 84 extends upward toward the delivery tube 420. The tabs are configured such that they remain engaged with the region 84 after the delivery unit is removed.

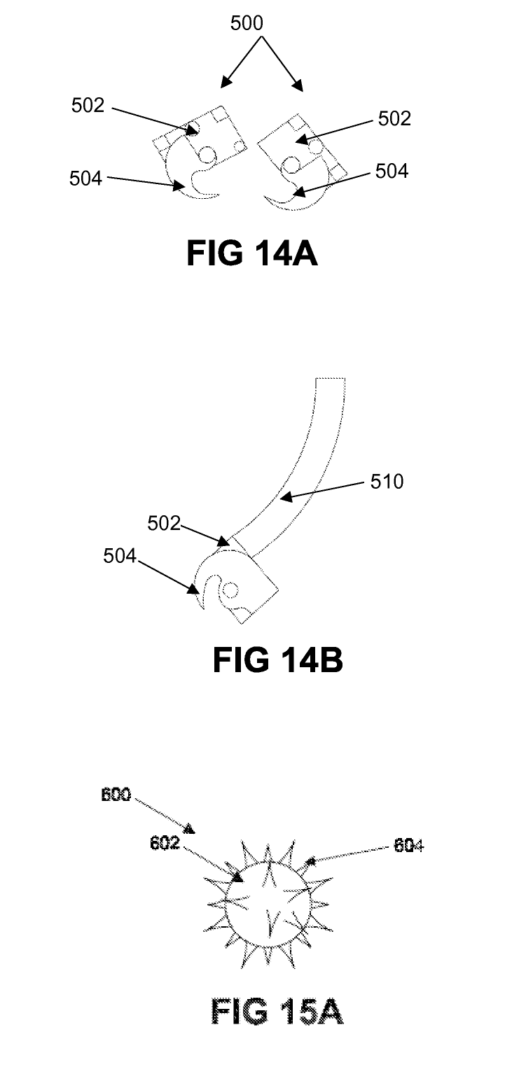

FIG. 14A depicts a side view of a marker 500. The marker 500 includes at least one hooking unit 504 coupled to a base unit 502. The hooking unit 504 is a substantially “C” shaped unit that is connected to the base unit 502 at two points, though the hooking unit 504 may be connected to the base unit 502 by one or any number of points. FIG. 14B depicts a marker 500 connected to a delivery unit 510. The delivery unit 510 forces the hooking unit 504 into a region by applying a force perpendicular to the base unit 502 of the marker 500.

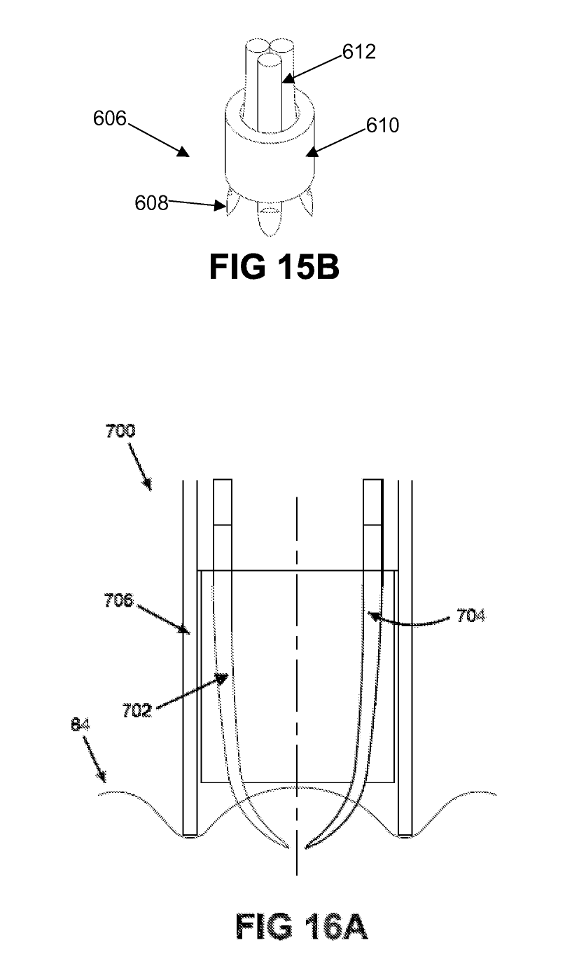

FIG. 15A depicts a perspective view of a marker 600. The marker 600 includes a spherical base 602 with a plurality of tapered cylindrical units 604 adhered to the surface of the base 602. In one embodiment, the spherical base 602 is color coded to indicate the location of the marker 600. In another embodiment, the circular base 602 includes an RFID tag to indicate the location of the marker 602. In another embodiment, the circular base 602 includes a unique letter, number or symbol on the top surface of the circular base. The tapered cylindrical units 604 have sharp pointed ends configured to adhere to the surface of a region.

FIG. 15B depicts a perspective view of a delivery unit 606 for the marker 600. The delivery unit 606 includes at least three holding units 608 arranged around the marker 600 and a sleeve 610 positioned around the marker 600. The sleeve 610 is slightly larger than the marker 600 such that the sleeve fits around the exposed portions of the marker 600 when the marker 600 is embedded in a region 84. Each of the holding units 608 are connected to an extension unit 612. Prior to the implantation of the marker 600 in the region 84, the sleeve 610 is positioned around the marker 600 and the extension units 612. The holding units 608 are configured to hold the marker 600 such that the marker 600 is not free to move. During deployment, the extension units 612 force the lower portion of the marker 600 into the region 84 such that the marker 600 is embedded into the region 84. Next, the extension units 612 are pulled away from the region 84 through the sleeve 610 leaving the marker 600, including the holding units 608 and the sleeve 610 in the region 84.

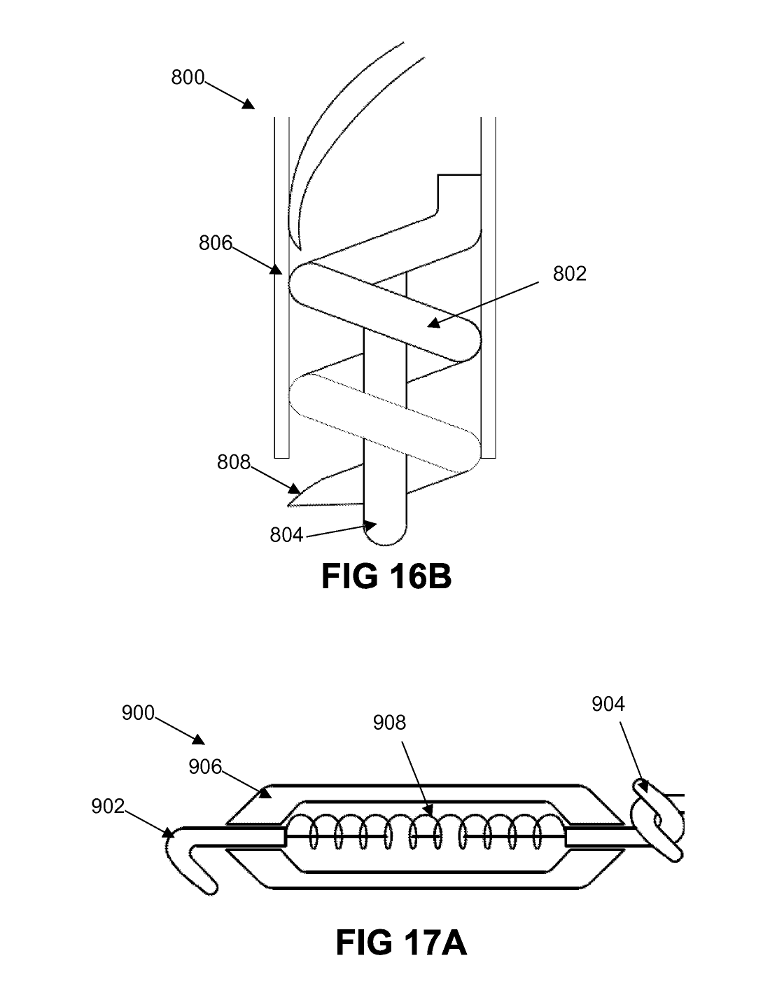

FIG. 16A depicts a side view of a marker 700. The marker 700 includes at least two hooking units 702 and 704 in a protective casing 706 that includes at least one opening. The hooking units 702 and 704 are curved towards the center of the casing 706 and are initially separated by a distance such that the ends of the hooking units are not in contact. In addition, the lower portions of the hooking units 702 and 704 extend beyond the opening in the protective casing 706. During deployment, the hooking units 702 and 704 are forced towards the region 84 by a vertical transmission unit (not shown). When the ends of the hooking units 702 and 704 enter the region 84, the hooking units 702 and 704 move laterally towards each other until the ends of the two hooking units are in contact. In one embodiment, the protective casing is color coded to indicate the location of the marker. In another embodiment, the protective case includes an RFID tag to indicate the location of the marker.

FIG. 16B depicts a side view of a marker 800. The marker 800 includes a screw 802 wound around a base unit 804. The screw 802 is adhered to a base unit 804 and includes a sharpened pointed end 808 capable of puncturing a region 84 (not shown) that extends beyond an open end of the protective case 806. During deployment, the sharpened end 808 of the screw 802 penetrates into a portion of a region 84. A rotational force is then applied to the screw 802 such that the screw travels into the region 84 to secure the marker 800 into the region 84. It is understood that the base unit 804 may be attached to a cap unit (not shown) or the base unit 804 may extend above and out of the screw 802 and that the base unit 804 may itself mark a site within region 84. In one embodiment, the protective case 806 is color coded to indicate the location of the marker 800. In another embodiment, the protective case 806 includes an RFID tag to indicate the location of the marker 800. In another embodiment, the protective case 806 includes a unique letter, number or symbol on the surface of the protective case 806.

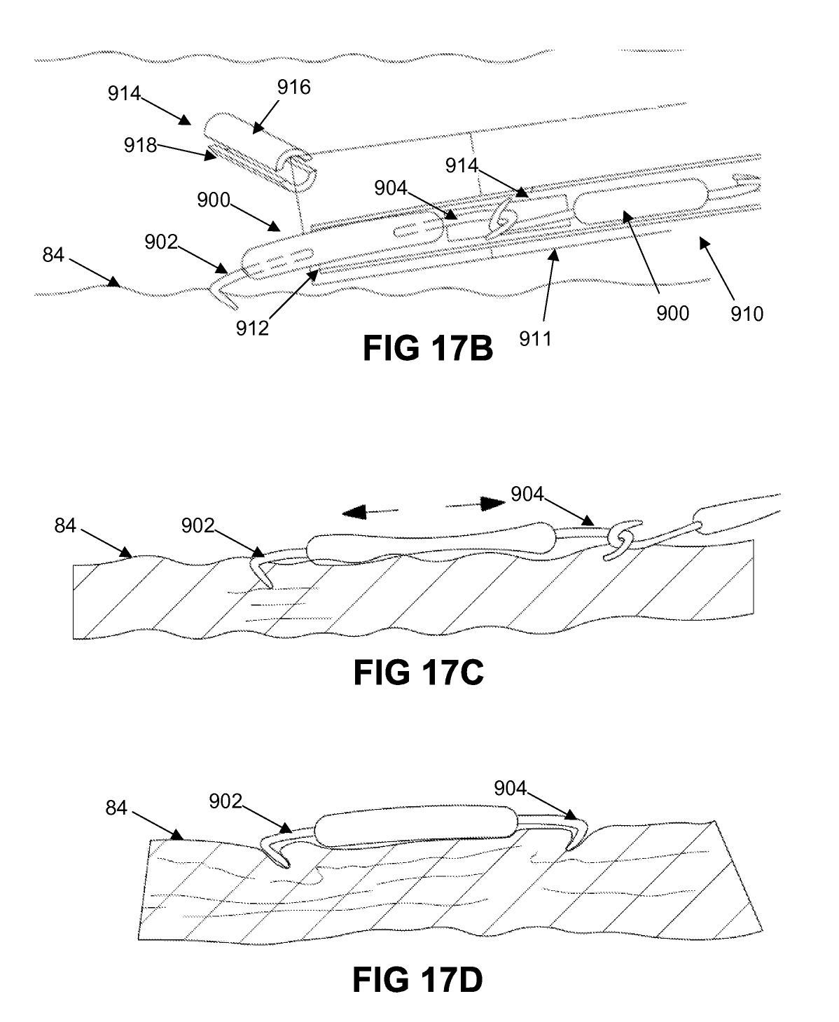

FIG. 17A depicts a side view of a marker 900. The marker 900 includes a first hooking unit 902 having one curved end and one substantially straight end, a second hooking unit 904 having one curved end and one substantially straight end and a protective case 906 covering the straight ends of the hooking units 902 and 904. The straight ends of the hooking units 902 and 904 are coupled by a spring unit 908. The spring unit 908 applies a force to the straight ends of the hooking units 902 and 904 such that the straight ends of the hooking units are pulled towards each other. Further, the protective case 906 covers the spring unit 908 entirely. In one embodiment, the protective case 906 is color coded to indicate the location of the marker 900. In another embodiment, the protective case 906 includes an RFID tag to indicate the location of the marker 900. In yet another embodiment, the protective case 906 has a unique letter, number or symbol on the top surface of the protective case 906.

FIG. 17B depicts a side view of a delivery unit 910 for the marker 900. The delivery unit 910 is comprised of a tube 911 having an opening 912. A plurality of markers 900 are stacked in the delivery unit 910 such that the second hooking unit 904 of one marker 900 is coupled to the first hooking unit 902 of another marker 900. A spacer 914 covers the coupled first and second hooking units. In one embodiment, the spacer 914 consists of an upper portion 916 and a lower portion 918 which are separate from one another and are held together by the walls of the tube 911. When the spacer 914 exits the tube 911 the upper portion 916 and lower portion 918 separate from one another and fall off of the marker 900. The marker 900 closest to the opening 912 extends through the opening such that the first hooking unit 902 of the marker 900 is exposed with a pointed end of the hooking unit 902 facing the region 84.

FIG. 17C depicts a side view of a marker 900 engaging a region 84. The first hooking unit 902 of the marker is embedded into the region 84 by a downward force applied by the delivery unit 910 that forces the sharpened portion of the first hooking unit 902 into the region 84. After the first hooking unit 902 is secured to the region 84, the delivery unit 910 moves away from the hooking unit 902 thereby pulling the second hooking unit 904 away from the first hooking unit 902 and extending the spring unit 908. Second hooking unit 904 moves away from the first hooking unit 902 until the force of the spring unit 908 is sufficient to pull the second hooking unit 904 free from the first hooking unit 902 of the next marker 900 in the tube 911. Once detached, the spring unit 908 pulls the second hooking unit 904 towards the first hooking unit 902 such that the second hooking unit becomes embedded into the region 84 as shown in FIG. 17D.

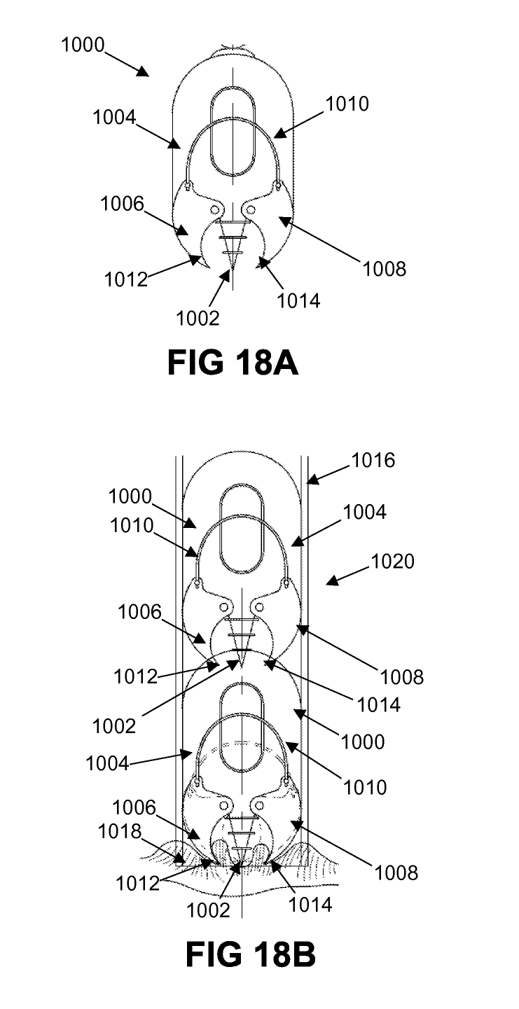

FIG. 18A depicts a side view of a marker 1000. The marker 1000 includes a barb unit 1002 affixed to a protective case 1004. Two securing units 1006 and 1008 are rotatively affixed to protective case 1004 on both sides of the barb unit 1002. The securing units 1006 and 1008 are comprised of hooking portions 1012 and 1014 which extend from the protective case 1004 such that the end of the barb unit 1002 extends slightly beyond the ends of the securing units 1006 and 1008. The upper portions of the securing units 1006 and 1008 are coupled together by a curved wire 1010. The curved wire 1010 applies a forced to the securing units which cause the hooking portions 1012 and 1014 to move towards the barb unit 1002 when the marker is engaged with a region.

FIG. 18B depicts a side view of a delivery unit 1020 for the marker 1000. The delivery unit 1020 includes a tube 1016 that has an opening 1018 at one end of the tube 1016. A plurality of markers are stacked in the delivery tube such that the top portion of a first marker 1000 that is closer to the opening 1018 is in contact with the lower portion of a second marker 1000 located above the first marker. A vertical transmission unit (not shown) exerts a downward force concentrated on the top of the protective case 1004 of each marker causing the plurality of markers to move downward in the opening 1018. In addition, the securing portions 1012 and 1014 of each marker are in contact with the sidewalls of the tube 1016 which cause the wire 1010 connecting the securing portions 1006 and 1008 to compress.

As the markers 1000 are forced down the tube 1016, the barb unit 1002 of the marker closest to the opening 1018 penetrates the region 84. As the marker 1000 continues to move towards the region 84, the barb unit 1002 further penetrates the region 84 until the securing units 1006 and 1008 are free of the tube 1006. When the securing units 1006 and 1008 are free of the tube 1016, the curved wire 1010 connecting the securing units 1006 and 1008 expands and moves towards the region 84 which causes the securing units 1006 and 1008 to rotate such that the hooking portions 1012 and 1014 rotate towards the barb unit 1002. When the hooking portions 1012 and 1014 move towards the barb unit 1002 they each force portions of the region 84 against the barb unit 1002 securing the barb unit in place. It is understood that other mechanisms than the curved wire 1010 may be used to rotate the securing units 1006 and 1008. In one embodiment, the protective case 1004 is color coded to indicate the location of the marker 1000. In another embodiment, the protective case 1004 includes an RFID tag to indicate the location of the marker 1000. In yet another embodiment, the protective case 1004 has a unique letter, number or symbol on the top surface of the protective case 1004.

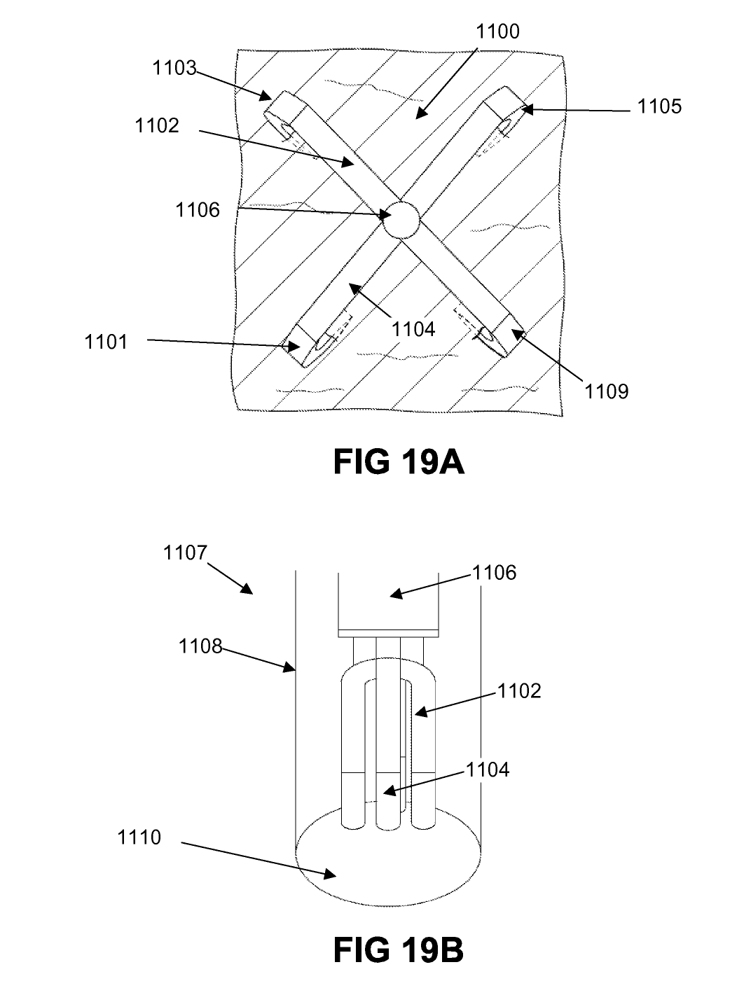

FIG. 19A depicts a top view of a marker 1100. The marker 1100 comprises a first hooking unit 1102 and a second hooking unit 1104. The ends of the hooking units 1102 and 1104 include curved latching units 1101, 1103, 1105 and 1109 which curve inward towards the center of each hooking unit 1102 and 1104 to create a hook shape. The curved latching units 1101, 1103, 1105 and 1109 are rotatively connected to the ends of the hooking units 1102 and 1104 by springs which apply a downward force on the curved latching units 1101, 1103, 1105 and 1109. Further, the curved latching units 1101, 1103, 1105 and 1109 are connected to a pin 1106 located in the center portion of the marker which pulls the curved latching units 1101, 1103, 1105 and 1109 towards the pin and which also connects the hooking units 1102 and 1104 together.

FIG. 19B depicts a side view of a delivery unit 1107 for the marker 1100. The delivery unit 1107 includes a tube 1108 that has an opening 1110 at one end. A plurality of markers are stacked in the tube 1108 such that the pin 1106 of a marker closer to the opening 1110 is in contact with another marker or with a spacer placed in between the individual markers. The hooking units 1102 and 1104 are arranged in the tube 1108 such that the ends of the first hooking unit 1102 oppose each other and the ends of the second hooking unit 1104 oppose each other. A vertical transmission unit (not shown) forces the markers to move down the tube towards a region 84.

When the hooking units 1102 and 1104 extend beyond the tube, the hooking units 1102 and 1104 extend parallel with the region 84. The delivery unit continues to apply a downward force until the lower portion of the pin 1106 creates an indentation in the region 84 and forces the ends of the hooking units 1102 and 1104 to bend away from the region. The delivery unit 1107 pulls the pin 1106 away from the region 84 which forces the hooking units 1102 and 1104 to move towards the region 84. As the pin 1106 moves away from the region 84 while remaining attached to the marker, hooking units 1102 and 1104 and the curved latching units 1101, 1103, 1105 and 1109 move towards the region 84. As the pin 1106 is pulled away from the region 84, the springs connected to the curved latching units 1101, 1103, 1105 and 1109 are released which forces the curved latching units 1101, 1103, 1105 and 1109 downward into the region 84 as shown in FIG. 19C. In one embodiment, the pin 1106 is color coded to indicate the location of the marker 1100. In another embodiment, the pin 1106 includes an RFID tag to indicate the location of the marker 1100. In another embodiment, the top surface of the pin 1106 includes a unique letter, number or symbol.

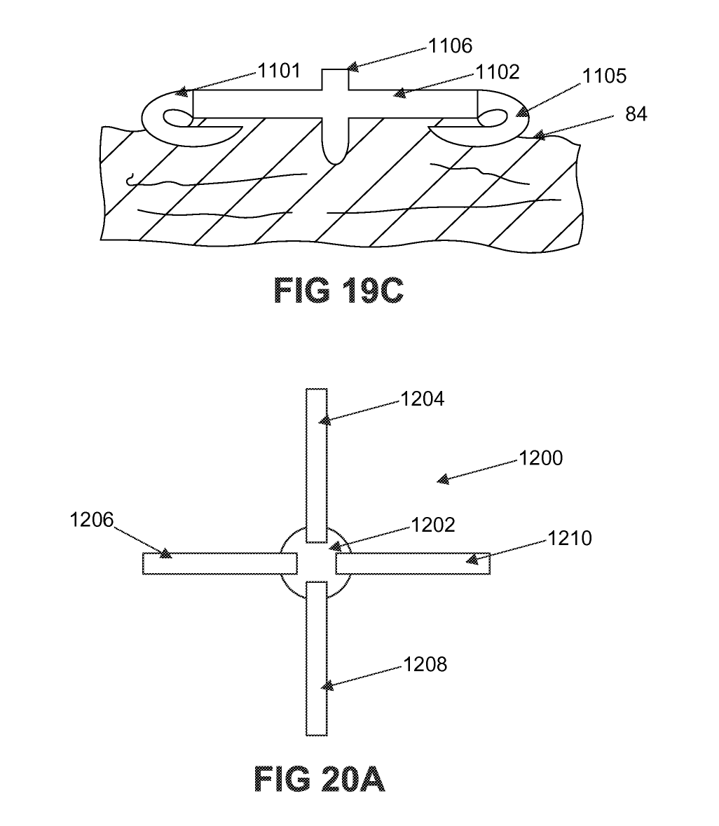

FIG. 20A depicts a top view of another embodiment of a marker 1200. The marker 1200 includes a center disk 1202 and at least four extension arms 1204, 1206, 1208 and 1210. The extension arms 1204, 1206, 1208 and 1210 are rotatively connected to the center disk 1202 by a hinge or a spring such that the extension arms 1204, 1206, 1206 and 1210 are forced downward towards a region.

FIG. 20B depicts a side view of a delivery unit 1212 for the marker 1200. The delivery unit 1212 includes a tube 1214 that has an opening 1216 at one end. The extension arms 1204, 1206, 1208 and 1210 (not shown) of the marker are positioned such that the unsecured end of each extension arm is above the center disk 1202. The spring units that connect the extension arms 1204, 1206, 1208 and 1210 to the center disk apply a downward force on the extension arms such that pins 1218, 1220, 1222 and 1224 (not shown in Figure) are pressed against the interior walls of the tube 1214. A plurality of markers 1200 are stacked in the tube 1214 such that the lower portion of the center disk 1202 of an upper marker 1200 is in contact with the ends of the extension arms 1204, 1206, 1208 and 1210 of a lower marker.

A vertical transmission unit (not shown) pushes the marker 1200 downward such that the marker 1200 moves towards the opening 1216 of the tube 1214. As the marker 1200 moves downward towards the opening 1216, a pin 1226 positioned in the center of the lower portion of the center disk 1202 exits the tube and indents the region 84. The marker 1200 continues to move downward in tube 1214 pushing the pin 1226 into the region 84 until the extension arms 1204, 1206, 1208 and 1210 become free of the tube and are forced downward towards the region 84 by the spring units. The spring units force the pins 1218, 1220, 1222 and 1224 into the region 84 as shown in FIG. 20C. In another embodiment, the pins 1218, 1220, 1222, 1224 and 1226 are barbed. In one embodiment, the center disk 1202 is color coded to indicate the location of the marker 1200. In another embodiment, the center disk 1202 includes an RFID tag to indicate the location of the marker 1200. In another embodiment, the top surface of the center disk 1202 includes a unique letter, number or symbol.

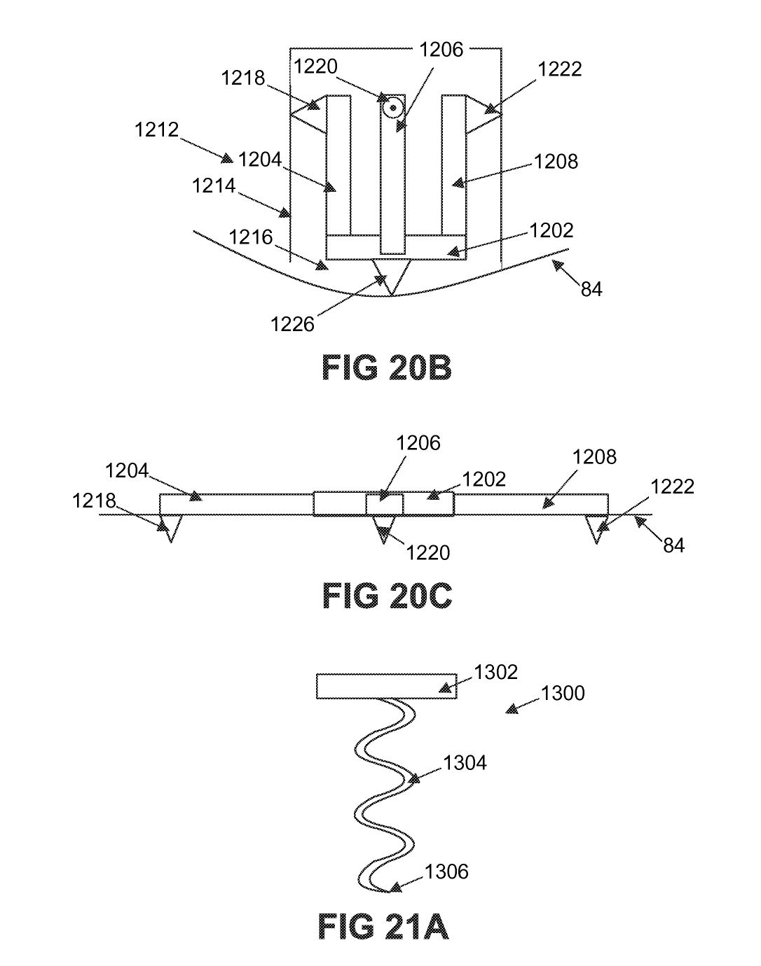

FIG. 21A depicts a side view of a marker 1300. The marker 1300 includes a cap unit 1302 and a screw shaped unit 1304 having one end affixed to the center of the lower portion of the cap unit 1302 and another end 1306 sharpened to a point capable of puncturing a region (not shown). In one embodiment, the cap unit 1302 is color coded to indicate the location of the marker 1300. In another embodiment, the cap unit 1302 includes an RFID tag to indicate the location of the marker 1300.

FIG. 21 B depicts a delivery unit 1308 for the marker 1300. The delivery unit 1308 includes an inner tube 1310 that has an opening 1312 and an outer tube 1314 that shares the opening 1312 at one end. The markers 1300 are stacked in the inner tube 1310 such that the sharpened end 1306 of a marker is in contact with the cap unit 1302 of another marker closer to the opening 1312. In one embodiment, the inner tube 1310 is allowed to rotate freely in relation to the outer tube 1314 such that a rotational force is applied to the cap unit 1302 that forces the cap unit 1302 down towards the opening 1312. The rotational force applied by the inner tube 1310 forces the marker downward out of tube. As the marker 1300 moves downward towards the region, the sharpened point 1306 penetrates the region 84. The marker continues downward until the cap unit 1302 is free of the tube. However, any mechanism which allows for the transmission of a rotational force to the marker 1300 effecting its screw-type deployment into a region 84 will do.

FIG. 21C depicts a delivery unit 1308 for a marker 1300. As FIG. 21C depicts, the cap unit 1302 of the marker 1300 includes a plurality of grooves 1316 that engage at least one gear unit 1318. The inner sidewalls of tube 1320 also include a plurality of grooves 1322 which engage the teeth of the gear unit 1318 such that the top portion of the marker 1300 rotates as the marker 1300 is moved down the tube 1320. Once the marker 1300 is clear of the tube, the gears 1318 fall away. FIG. 21D depicts the marker 1300 embedded in the region 84.

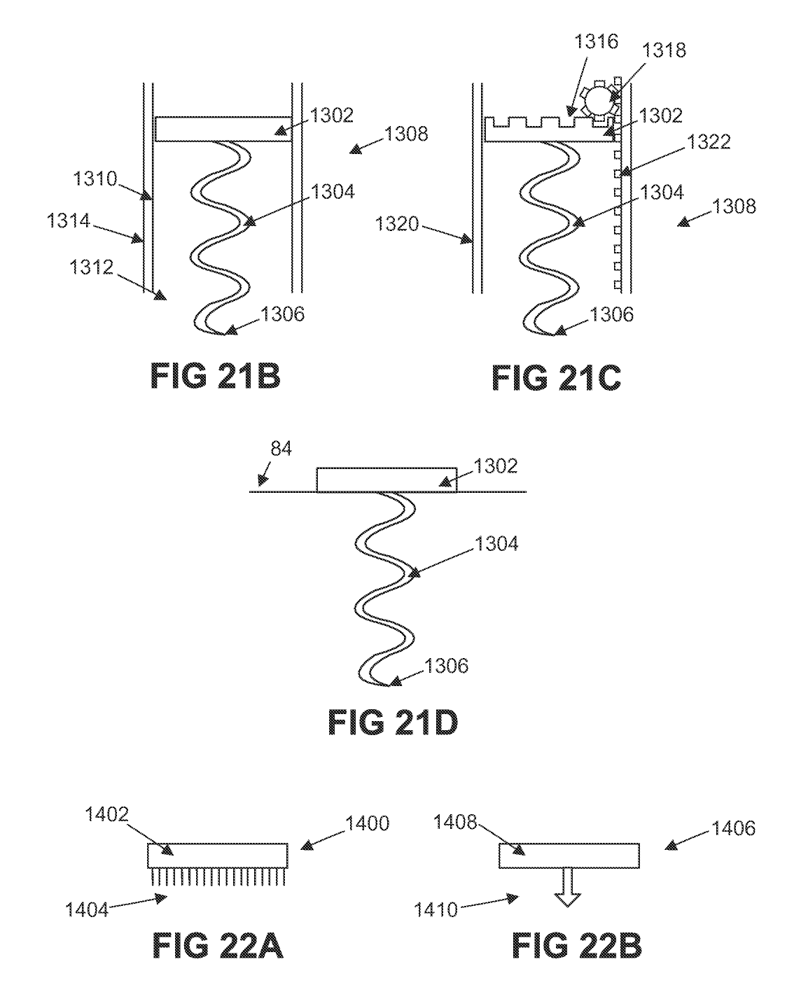

FIG. 22A depicts a side view of a marker 1400. The marker 1400 includes a cap unit 1402 and a plurality of micro-bristles 1404 affixed to the lower portion of the cap unit 1402. The micro-bristles 1404 engage the surface of a region 84 such that the micro-bristles secure themselves to the region 84. The micro-bristles 1404 may be substantially straight, curved, barbed or contain any other arrangement to aid in their securing the marker to the wall of the GI tract. In one embodiment, the cap unit 1402 is color coded to indicate the location of the marker 1400. In another embodiment, the cap unit 1402 includes an RFID tag to indicate the location of the marker 1400. In another embodiment, the cap unit 1402 includes a unique letter, number or symbol on the surface of the cap unit 1402.

FIG. 22B depicts a side view of a marker 1406. The marker 1406 includes a cap unit 1408 and an attachment unit 1410 affixed to the center of a lower portion of the cap unit 1408. In one embodiment, the marker 1406 may include a plurality of attachment units 1410 affixed to the lower portion of a cap unit 1408. The attachment units are formed into any shape which is capable of adhering to the wall of the GI tract. In another embodiment, the attachment unit 1410 is in the shape of a barb. In another embodiment, a plurality of attachment units are arrayed in any direction and/or orientation in relation to one another. In another embodiment, the attachment unit is a taper screw which has a base portion affixed to the cap unit 1408 which tapers to a point on the end opposite the cap unit 1408. In yet another embodiment, the tapered screw 1408 includes retractable pins which engage the region 84. In one embodiment, the cap unit 1408 is color coded to indicate the location of the marker 1406. In another embodiment, the cap unit 1408 includes an RFID tag to indicate the location of the marker 1406. In another embodiment, the cap unit 1408 includes a unique letter, number or symbol on the surface of the cap unit 1408.

FIG. 22C depicts a side view of a marker 1412. The marker 1412 includes a cap unit 1414 and an adhesive layer 1416 applied to the lower portion of the cap unit 1414. The adhesive layer 1416 is comprised of a non-toxic adhesive capable of creating a secure bond to human tissue. In one embodiment, the cap unit 1414 is color coded to indicate the location of the marker 1412. In another embodiment, the cap unit 1414 includes an RFID tag to indicate the location of the marker 1412. In another embodiment, the cap unit 1414 includes a unique letter, number or symbol on the surface of the cap unit 1414.

FIG. 22D depicts the side view of a marker 1418. The marker 1418 includes a cap unit 1420, at least three barbs 1422 aligned on the lower surface of the cap 1420 and at least two pins 1424 aligned on the lower surface of the cap 1420. In one embodiment, the barbs 1422 are comprised of a straight shaft having one end secured to the cap unit 1420 and a protrusion extending perpendicular to a side of the shaft at the end opposite the cap unit 1420. The end of the protrusion opposite the surface of the shaft forms into a sharpened point completing the barb 1422. In one embodiment, the hooking portion 1426 of two of the barbs are oppositely facing. However, the barbs 1422 may be aligned in any configuration that allows the marker unit to secure to a region 84 and may include any number or combination of barbs and pins.

FIG. 22E depicts a bottom view of the marker 1418. In one embodiment, the barbs 1422 are aligned along one axis of the lower portion of the surface. In addition, the pins 1424 are aligned along an axis perpendicular to the axis along which the barbs 1422 are aligned. FIG. 22F depicts a side view of an embodiment of the marker 1418. In this embodiment, the pins 1424 are replaced with two barbs 1428 having hooking portions that are facing away from the center of the cap 1420 and which are angled away from the center of the cap 1420.

In one embodiment, the cap unit 1420 is color coded to indicate the location of the marker 1418. In another embodiment, the cap unit 1420 includes an RFID tag to indicate the location of the marker 1418. In another embodiment, the cap unit 1420 includes a unique letter, number or symbol on the top surface of the cap unit 1420.

FIG. 22G depicts a side view of a marker 1430. The marker 1430 includes a plurality of barbs 1432 affixed to the lower portion of a cap unit 1434. The barbs 1432 include sharpened ends 1436 which face away from the center of the cap unit 1434. In one embodiment, the barbs 1432 are perpendicular to the lower surface of the cap unit 1434. In another embodiment, the barbs 1432 are angled away from the center of the cap unit 1434. In yet another embodiment, each of the barbs 1432 is angled in different directions relative to one another.

In one embodiment, the cap unit 1434 is color coded to indicate the location of the marker 1430. In another embodiment, the cap unit 1434 includes an RFID tag to indicate the location of the marker 1430. In another embodiment, the cap unit 1434 includes a unique letter, number or symbol on the top surface of the cap unit 1434.

FIG. 22H depicts a side view of a marker 1440. The marker 1440 consists of a plurality of barbs 1442 affixed to the lower surface of a cap unit 1444. The barbs 1442 are comprised of a shaft 1446 having one end affixed to the lower portion of the cap unit 1444 and a second end affixed to a top portion of a first pin 1448. The first pin 1448 includes an upper base portion and a lower pointed end. The shaft 1446 is offset from the center of the upper base portion of the first pin 1448 by a predetermined distance. A lower base portion of a second pin 1450 is affixed to the upper base portion of the first pin 1448 such that the sharpened end of the second pin 1450 faces a direction opposite or nearly opposite to the sharpened end of the first pin 1448. Further, the second pin 1450 is sized relative to the first pin 1448 such that a gap exists between the second pin and the shaft 1446. During deployment, the marker 1440 is extended downward into a region 84 (not shown) such that the first pin 1448 pierces the region 84 and extends into the region. The marker 1440 continues into the region 84 until the second pin 1450 is below the surface of the region 84. Because of this arrangement, the second pin 1450 prevents the marker 1440 from exiting the region 84. In another embodiment, the first pin 1448 and/or the second pin 1450 may be blunt instead of sharp. In another embodiment, the cap unit 1444 may have one or a plurality of holes or gaps that allow for passage of either a needle or a blunt-tipped stylet or a plurality of either needles and/or blunt-tipped stylets in order to aid in the deployment of a marker 1440 onto and/or into a region 84.

In one embodiment, the barbs 1442 are perpendicular to lower surface of the cap unit 1444. In another embodiment, the barbs 1442 are angled away from the center of the cap unit 1444. In yet another embodiment, each of the barbs 1442 are angled in different directions relative to one another. In each embodiment, the barbs may face in the same or different directions and multiple barbs may be affixed to the lower portion of the cap unit.

In one embodiment, the cap unit 1444 is color coded to indicate the location of the marker 1440. In another embodiment, the cap unit 1444 includes an RFID tag to indicate the location of the marker 1440. In another embodiment, the cap unit 1444 includes a unique letter, number or symbol on the top surface of the cap unit 1444.

The markers 1400, 1406, 1412, 1418, 1420, 1430 and 1440 are inserted into a delivery unit such that the upper portion of the cap units are in contact with the lower portions of another marker. A vertical transmission unit (not shown) applies a force to the center of the cap units such that the cap unit closest to the region 84 is pushed out of the delivery unit and onto the surface of the region 84 thereby allowing the entire marker to be secured to the region 84. In an alternative embodiment, a spacer is employed as part of the delivery unit.

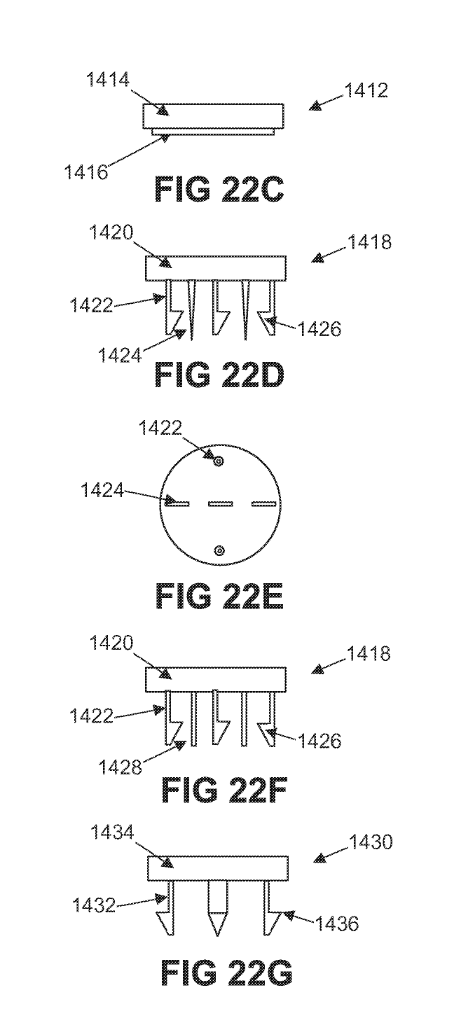

FIG. 23A depicts a top view of a marker 1500. The marker 1500 includes a center unit 1502 that includes four retractable barbs 1504, 1506, 1508 and 1510. Alternatively, the center unit 1502 may include two, three, or more than four barbs. Initially, the barbs 1504, 1506, 1508 and 1510 are retracted into a center portion of the center unit 1502. After the marker 1500 is positioned on the region 84, an engagement unit 1512 is depressed which forces or releases the barbs 1504, 1506, 1508 and 1510 out of the center portion and into the region. In one embodiment, the engagement unit 1512 is coupled to a shaft that forces the barbs 1504, 1506, 1508 and 1510 out of the center unit 1512 when the engagement unit 1512 is depressed. Consistent with this embodiment, the center unit 1502 may be tapered such that the thickness of the center unit 1502 increases from the bottom of the marker to the top of the marker. In one embodiment, the center unit 1502 is color coded to indicate the location of the marker 1500. In another embodiment, the center unit 1502 includes an RFID tag to indicate the location of the marker 1500. In another embodiment, the center unit 1502 includes a unique letter, number or symbol on the top surface of the center unit 1502.

FIG. 23B depicts a side view of a delivery unit 1514 for the marker 1500. The delivery unit 1514 includes a tube 1516 that has an opening 1518 at one end. A plurality of markers 1500 are stacked in the delivery unit 1514 with the barbs retracted into a center portion of the center unit 1502. The markers are forced towards the opening 1518 by a vertical transmission unit (not shown). As one marker exits the delivery unit 1514 through the opening 1518, the marker 1500 indents the region 84. With the marker 1500 indenting the region 84, the engagement unit 1512 is depressed which extends the barbs into the sides of the indented region 84.

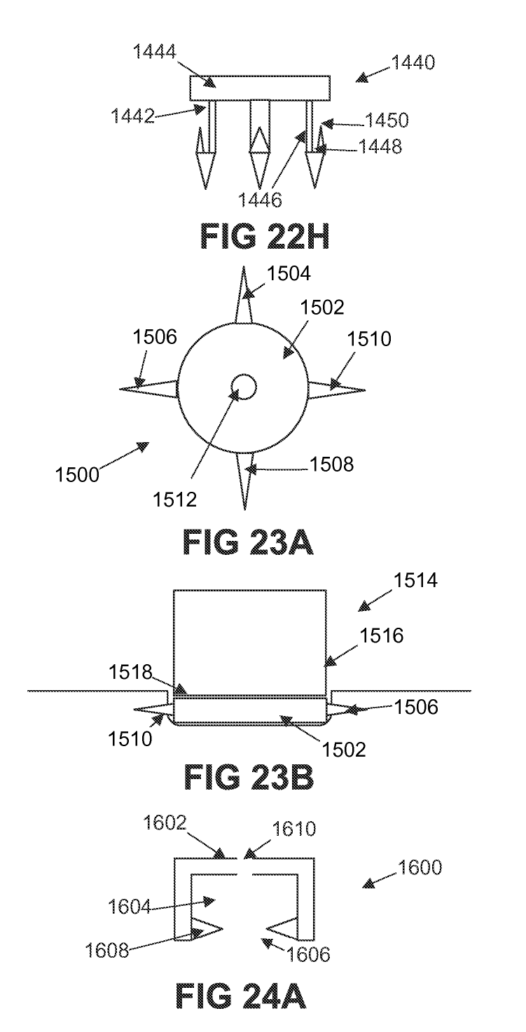

FIG. 24A depicts a cut away side view of a marker 1600. The marker 1600 includes a cap unit 1602 which includes a cavity 1604 in the center portion of the cap unit 1602 and an opening 1606 in the lower portion of the cap unit 1602. The sides of the opening 1606 include a plurality of barb units 1608. The marker 1600 also includes a second opening 1610 in the center of the top portion of the cap unit 1602. In one embodiment, the cap unit 1602 is color coded to indicate the location of the marker 1600. In another embodiment, the cap unit 1602 includes an RFID tag to indicate the location of the marker 1600. In another embodiment, the cap unit 1602 includes a unique letter, number or symbol on the top surface of the cap unit 1602.

FIG. 24B depicts a delivery unit 1612 for the marker 1600. The delivery unit 1612 includes a suction tube 1614 that has an opening 1616. The marker 1600 is held in the opening 1606 such that the lower portion of the marker 1600 extends through the opening 1616. The sides of the marker 1600 are pressed against the inner walls of the suction tube 1614 such that the inner walls create an air tight barrier with the sides of the marker 1600 when suction is applied to the upper portion of the marker 1600. After the marker is placed on the region 84, a suction is created in the tube 1614 causing a portion of the region 84 to extend up into the cavity 1604. When the suction is stopped, the barb units 1608 hold the portion of the region 84 in the cavity 1604. In another embodiment, the suction causes the barb units 1608 to retract against a spring in the cap unit 1602 such that the barbs 1608 pull away from the region 84. After the suction is removed, the barbs 1608 are forced into the region 84 by the spring. The barbs 1608 are oriented horizontally in this figure; however, it is understood that these barbs may be oriented diagonally upward, diagonally downward, or even vertically downward, or any combination of these orientations. Furthermore, it is understood that the second opening 1610 may be of any size in relation to the total diameter of the cap unit 1602. In another embodiment, the second opening 1610 is omitted, and the opening 1616 is relatively larger allowing the suction to be transmitted through this opening 1616. In yet another embodiment, the second opening 1610 is present, and the opening 1616 is also larger, allowing suction to be transmitted through both openings. It is also understood that the cap unit 1602 may be of any shape or figure and is not in any way restricted to having a circular shape.

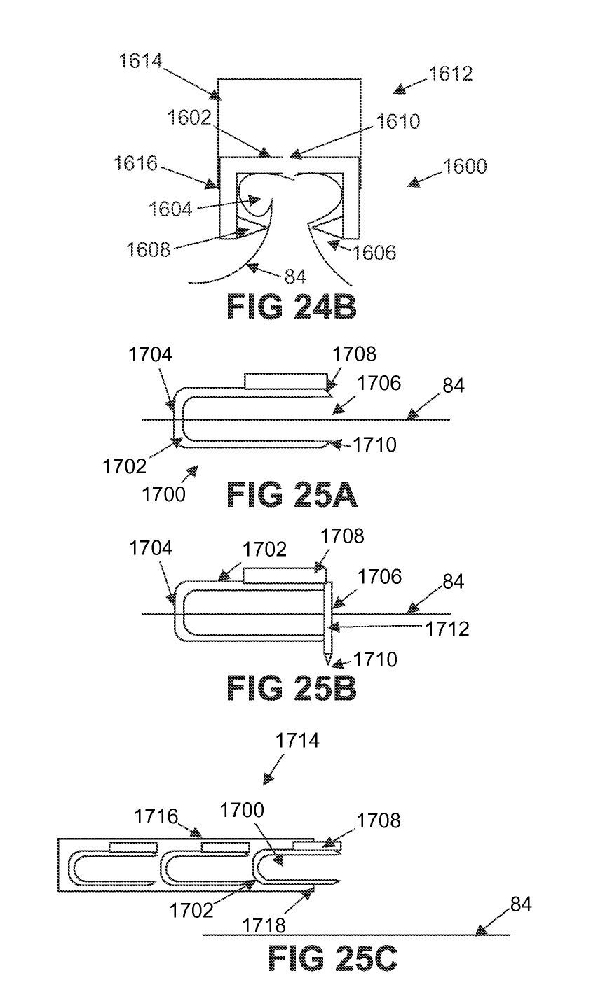

FIG. 25A depicts a side view of a marker 1700. The marker 1700 consists of a wire 1702 (or any other suitable material or object) bent into a substantially “U” shape having a closed end 1704 and an open end 1706. The open end 1706 includes a sharpened end 1710 and an unsharpened end which is affixed to the lower portion of a cap unit 1708. During engagement, the sharpened end 1710 penetrates the region 84 and extends parallel to the surface of the region 84.

FIG. 25B depicts a side view of the marker 1700 with a locking unit 1712. The locking unit 1712 secures the open end 1706 of the marker 1700 such that the marker 1700 does not move. In addition, the locking unit 1712 covers the sharpened end 1710 of the marking unit 1700. In another embodiment, two securing wires are affixed to an outer portion of the lower surface of the cap unit 1708 such that the securing wires extend downward towards the region and are parallel to one another on opposing sides of the cap unit 1708 preventing the marker 1700 from moving. In one embodiment, the cap unit 1708 is color coded to indicate the location of the marker 1700. In another embodiment, the cap unit 1708 includes an RFID tag to indicate the location of the marker 1700. In another embodiment, the cap unit 1708 includes a letter, number or symbol on the surface of the cap unit 1708. In yet another embodiment, the locking unit is omitted. In yet another embodiment, the securing wires are omitted. In another embodiment, both the locking unit and the securing wires are omitted.

In another embodiment, the cap unit 1708 of the marker 1700 is omitted and the portion of the “U” shaped wire 1702 opposite the sharpened end 1710 is formed into a longitudinally shaped marker unit. Consistent with this embodiment, the sharpened end 1710 of the marker 1700 penetrates a region 84. Further, the longitudinally shaped marker unit may be color coded, contain an RFID tag or include a number, letter or symbol on the surface of the wire. Any characters on the marker unit may be indented, notched, etched, printed, written with laser, or by any other means.

FIG. 25C depicts a delivery unit 1714 for the marker 1700. The delivery unit 1714 includes a tube 1716 that has an opening 1718 at one end. A plurality of markers 1700 are stacked in the delivery unit 1714 such that the closed end 1704 of one marker is in contact with the open end 1706 of another marker 1700. The markers are forced towards the opening 1718 by a vertical transmission unit (not shown) that applies a vertical force to the closed end 1704 of the wire. During deployment, the tube 1716 is positioned substantially parallel to the region 84 where the marker 1700 will be inserted. The vertical transmission unit (not shown) pushes the marker out of the tube 1716. In addition, the tube 1716 is tilted such that the sharpened end 1710 of the marker pierces the region 84. The vertical transmission unit (not shown) continues to force the marker 1700 into the region 84 until the marker 1700 is free of the tube 1716.

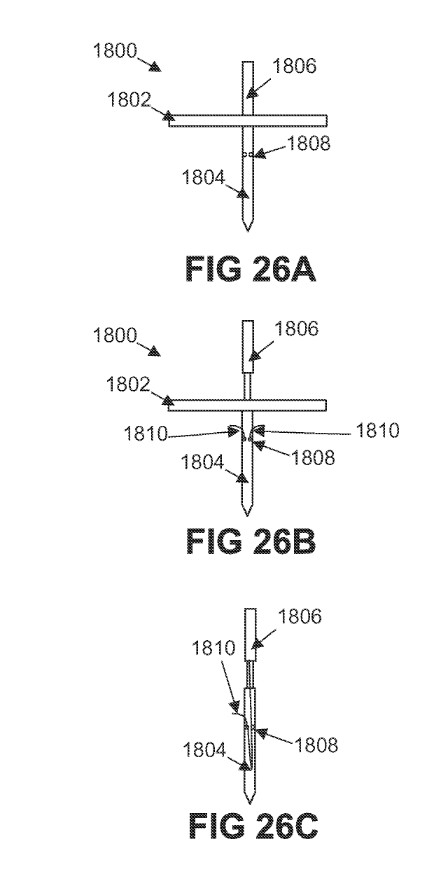

FIG. 26A depicts a side view of a marker 1800 that is consistent with the present embodiment. The marker 1800 includes a cap unit 1802, a pin unit or vertical transmission unit 1804 and an insertion unit 1806. The pin unit 1804 is affixed to the lower surface of the cap unit 1802 and is connected to the insertion unit 1806 through a hole in the center of the cap unit 1802. A lower portion of the insertion unit 1806 is connected to a plurality of wires 1810 inside the pin unit 1804. The wires 1810 are arranged such that they retract into the pin unit 1804 when the lower portion of the insertion unit 1806 extends into the pin unit 1804 and the wires 1810 extend out of openings 1808 in the pin unit 1804 when the insertion unit 1806 is pulled away from the pin unit 1804.

FIG. 26B depicts a side view of a marker 1800. The insertion unit 1806 is pulled away from the pin unit or vertical transmission unit 1804 which cause the wires 1810 to extend out from the opening 1808 in the pin unit 1804. In one embodiment, the wires 1810 are curved towards the pin unit 1804. In another embodiment, the wires 1810 are curved away from the pin unit 1804. In yet another embodiment, the wires 1810 are substantially straight and extend perpendicular from the openings 1808. In yet another embodiment, the wires 1810 are substantially straight and extend through the openings 1808 towards the cap unit 1802. However, the wires 1810 can be positioned in any direction or combination of directions as well as in any size, length, and/or shape sufficient to engage a region after the marker 1800 is inserted.

FIG. 26C depicts a side view of the connection between the insertion unit 1806 and the wires 1810. As the Figure depicts, each wire 1810 is curved such that an upward motion of the insertion unit 1806 translates into an upward movement of the wire 1810. In addition, a downward motion of the insertion unit 1806 translates into a downward motion of the wire 1810 which results in the wires 1810 retracting back into the pin unit or vertical transmission unit 1804. In another embodiment, the wires 1810 may point in any direction and different wires may point in different other directions. In another embodiment, the wires 1810 may be barbs or any other structures, or any combination of wires, barbs, and/or other structures which allow for anchoring of the marker 1800 into the GI wall. In yet another embodiment, all or some of the wires, barbs, and/or other structures will already be exposed prior to deployment of the marker 1800. In the event that all of the wires, barbs, and/or other structures are already exposed prior to deployment, the marker 1800 will not possess an insertion unit 1806

In one embodiment, the marker 1800 is extended into a region 84 using a vertical transmission unit. The pin unit or vertical transmission unit 1804 of the marker penetrates the region 84 such that the openings 1808 are surrounded by the region 84. Vertical transmission unit 1804 may be a needle which pierces region 84. An operator of the marker can pull the insertion unit 1806 which causes the wires 1810 to extend through the openings 1808 into the region 84 securing the marker in place.

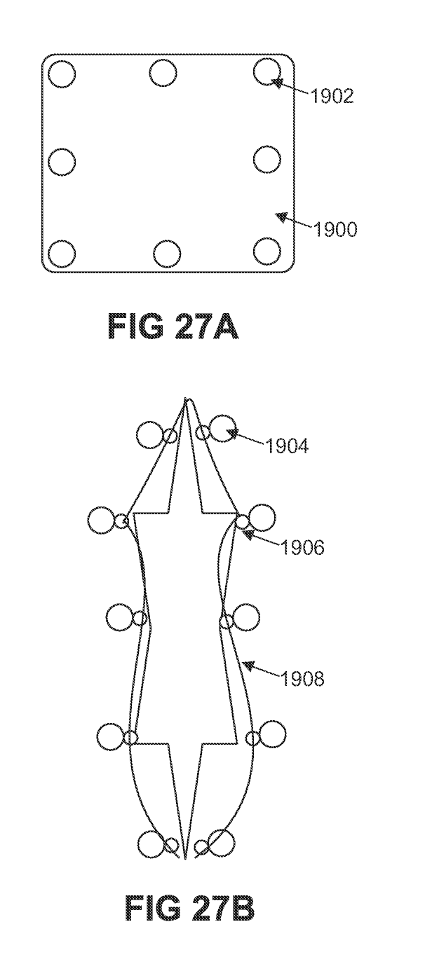

FIG. 27A depicts a top view of a material 1900 adhered to a region 84 using a plurality of markers 1902. Consistent with this embodiment, the markers used to adhere the material 1900 to the region 84 are any of the markers previously described. The material is any material that is non toxic to the human body. As an illustrative example, the material 1900 may be used to cover a tear or an opening in the region 84. The material 1900 is first positioned over the opening and is then adhered to the region 84 using a plurality of markers 1902 that are positioned in portions of the region 84 which can accommodate the markers. Alternatively, the material 1900 may include a bonding agent such as a glue or adhesive to adhere the material to the site within the region 84 or may be composed of a substance, or substances, that naturally bond, adhere and/or attach the material 1900 to the site within the region 84. The markers may be positioned as described above to further ensure that the material will not become displaced. In one embodiment, the material 1900 allows fluid, such as gas or a liquid, to pass through the material 1900 in one direction and restricts fluid from flowing through the material 1900 in the opposite direction. In one embodiment, the material 1900 covers an opening in a colon and the material 1900 allows gas and/or a liquid to flow into a colon and restricts gas and/or a liquid from flowing out of the colon.

FIG. 27B depicts a top view of a plurality of markers 1904. Each of the markers 1904 include a loop 1906 through which a suture 1908 is positioned. The loop 1906 is affixed to the cap unit of any of the previously described markers. The markers are connected to secure portions of the region 84. The suture 1908 is pulled through each loop 1906 such that both ends of the opening are pulled together to seal the opening. Once both ends are pulled together, the suture is tied together to secure the portions of the region 84 together. In one embodiment, the loop 1906 is recessed into the region 84 where the marker 1904 is inserted.

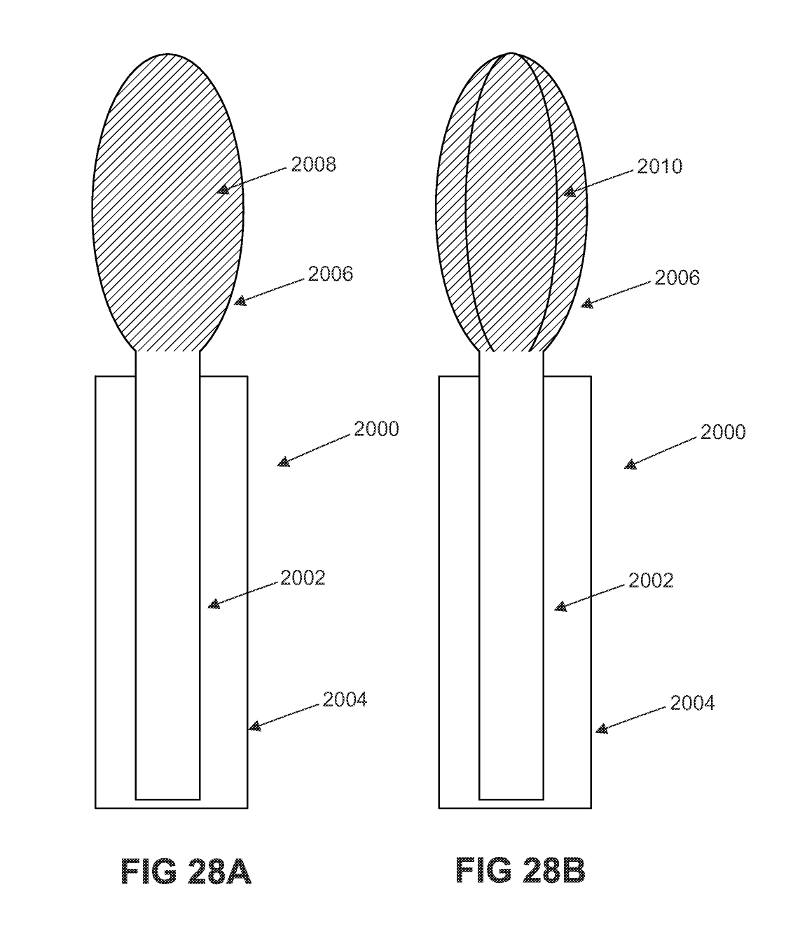

FIG. 28A depicts a top view of one embodiment of a mirror device 2000. The mirror device 2000 consists of an extension unit 2002 which retracts into a tube 2004. A vertical transmission unit (not shown) extends the extension unit 2002 out of the tube 2004. The end of the extension unit 2002 is formed into a mirror holding unit 2006. In one embodiment, the mirror holding unit 2006 has a substantially circular shape. In another embodiment consistent of the present embodiment, the mirror holding unit 2006 has a substantially elliptical shape.

A flexible reflective material 2008 is secured to the edges of the mirror holding unit 2006 which forms a flat reflective surface. The flexible reflective material 2008 has a reflective coating applied to one or both sides of the flexible reflective material 2008 such that light is reflected back from the surface of the flexible reflective material 2008. The edges of the mirror holding unit 2006 are formed of a material having memory capabilities that allow the mirror holding unit and the flexible reflective material 2008 to retract into the tube 2004. These shapes may be flat, concave or convex.

In one embodiment, the mirror device 2000 is used to position the mirror behind a growth on a region such that all sides of the growth are viewable. Additionally, the mirror device 2000 may be used to locate a finding or a previously placed marker device on the backside of a fold, or other structure, within the GI tract. It is understood, that the backside of a fold or structure is meant to mean a portion of the fold or structure out of direct view of an endoscopic device. In another embodiment, the mirror holding unit 2006 is rotatively attached to the extension unit 2004 which allows the mirror holding unit 2006 to rotate and bend in relation to the end of the tube 2004 such that different views of the growth are visible from the mirror. In one embodiment, the mirror holding unit 2006 is made from a material having memory capabilities such that the mirror holding unit 2006 contracts to fit into the tube 2004 when the vertical transmission unit (not shown) pulls the mirror holding unit 2006 into the tube 2004. In another embodiment, the mirror holding unit 2006 is made of a flexible material including, but not limited to, plastic, stainless steel, Teflon®, nitinol, nylon or any other material that is flexible with memory characteristics. In another embodiment, the mirror holding unit 2006 is made from a rigid material that is capable of retracting into the tube 2004 when a force acting to pull the mirror holding unit 2006 into the tube 2004 is applied.

In another embodiment the mirror holding unit 2006 expands into a basket 2010, as shown in FIG. 28B. In one embodiment, the basket 2010 expands to form the flexible reflective material 2008 into a convex lens. In another embodiment, the basket 2010 expands to form the flexible reflective material 2008 into a concave lens. In another embodiment, the flexible reflective material 2008 has a reflective coating on both sides which allows the basket 2010 to be used as a convex or concave mirror depending on the position of the mirror holding unit 2006. In another embodiment, the mirror holding unit 2006 expands into mirror having multiple reflective surfaces. In another embodiment, the mirror device is used to locate a previously placed marker in a region 84. In another embodiment, the mirror device is used to locate a region 84 in order to deploy a marker.

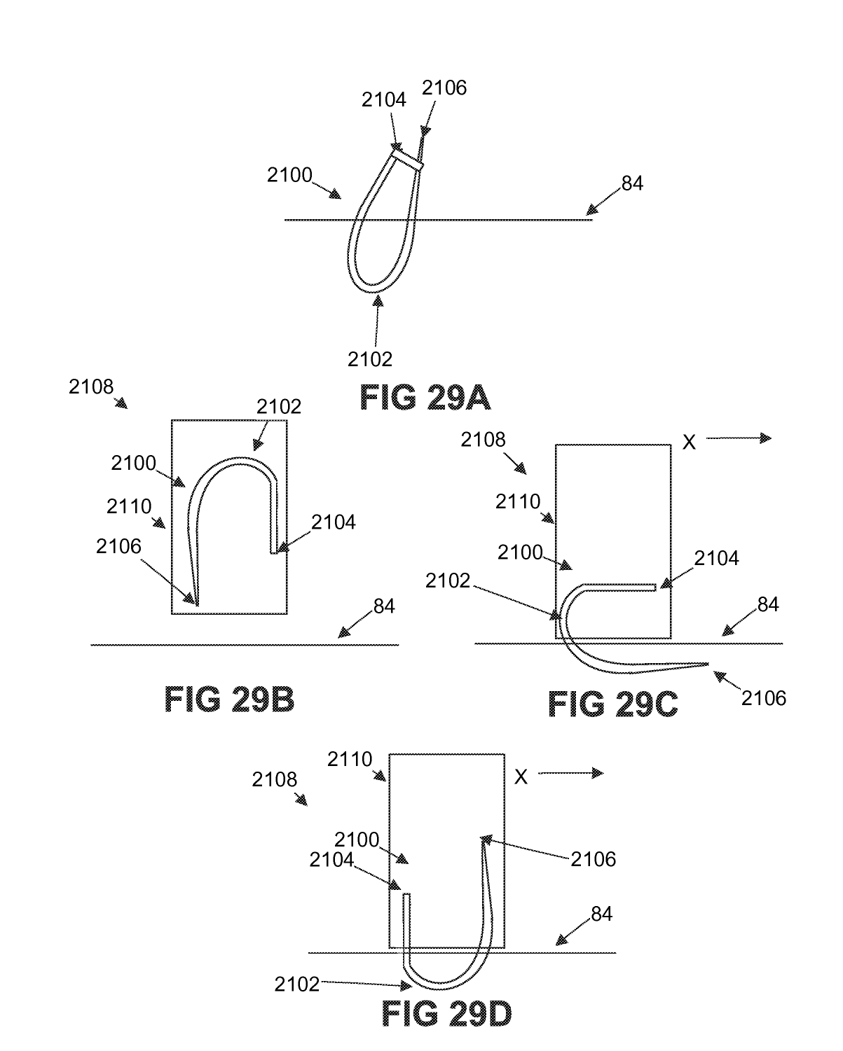

FIG. 29A depicts a side view of a marker 2100. The marker 2100 consists of a substantially “U” shaped loop unit 2102 that includes a latching unit 2104 rotatively affixed to a unsharpened end and a sharpened end 2106 opposite the unsharpened end. The sharpened end 2106 extends beyond the latching unit 2104 by a predetermined distance. The marker 2100 is comprised of a material having memory capabilities such that the material will attempt to return to a positioned where the unsharpened ends and sharpened ends are separated by a distance when no force is applied to the loop unit 2102.

During deployment, the sharpened end 2106 of the marker penetrates a first location of a region 84. The sharpened end 2106 moves through a portion of the region 84 such that the sharpened end 2106 extends through the surface of the region 84 at a second location separate from the first location. The sharpened end 2106 extends upward from the second location and is pulled toward the unsharpened end of the looping unit 2102 by an external force until the sharpened end extends through the latching unit 2104. The latching unit 2104 is then rotated over the sharpened end towards the region. The force on the sharpened end 2106 is removed and the sharpened end 2106 moves away from the unsharpened end such that the sharpened end 2106 is held against the latching unit 2104. In one embodiment, the latching unit 2104 includes a “J” shaped hook affixed to the end of the latching unit 2104 not secured to the unsharpened end of the loop unit. Consistent with this embodiment, the sharpened end rests in the “J” shaped hook after the external force is removed. In another embodiment, the sharpened end 2106 includes a notch located on the end facing the region 84 that allows the latching unit 2104 to rest in the notch when the latching unit 2104 is engaged with the sharpened end.

FIG. 29B depicts a delivery unit 2108 for the marker 2100. The delivery unit 2108 includes a tube 2110 which holds at least one marker 2100. As the Figure depicts, the marker 2100 is positioned in the tube such that the sharpened end 2106 of the marker 2100 points towards the region 84. As FIG. 29C depicts, the tube 2110 is lowered towards the region 84 while simultaneously being moved in a direction X As the marker 2100 and the tube 2110 move, the marker 2100 rotates such that the sharpened end 2106 rotates out of the tube 2110 and penetrates into the region 84. As the tube 2110 continues to move in the direction X, the marker 2100 moves through the region 84 until the sharpened portion of the marker 2100 extends upward out of the region 84, as depicted in FIG. 29D.

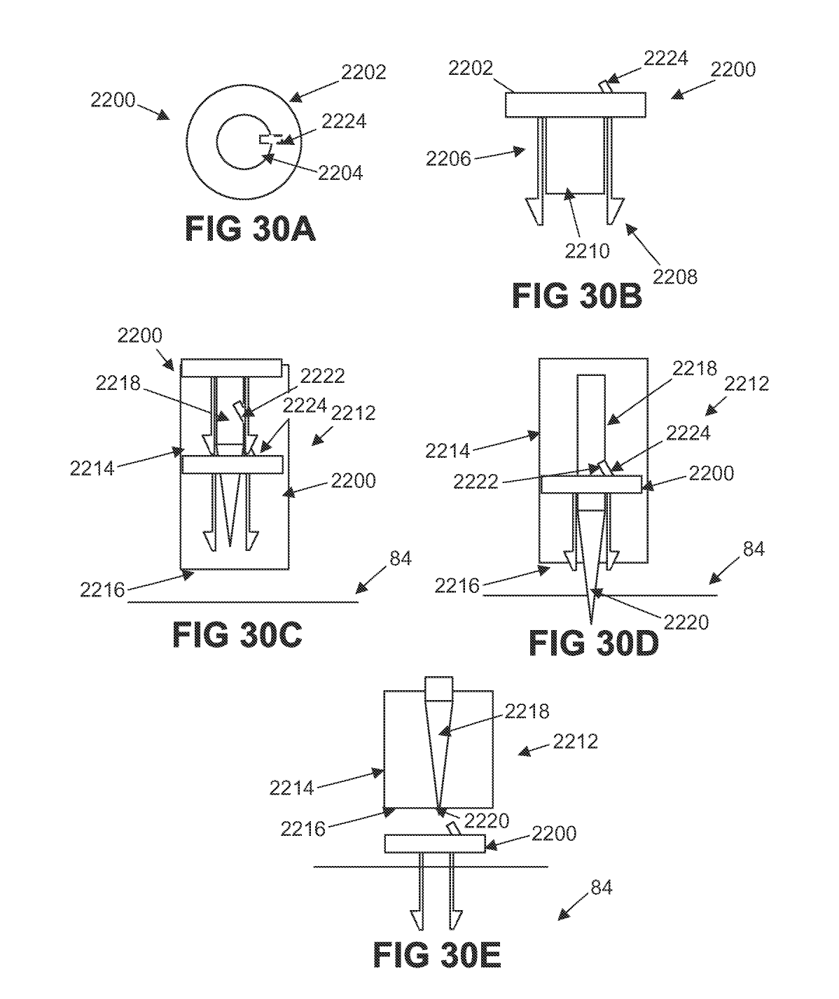

FIG. 30A depicts a top view of a marker 2200. The marker 2200 includes a cap unit 2202 with at least one opening 2204 and a spring loaded pin 2224. In one embodiment, the cap unit 2202 includes a plurality of openings 2204 located around the periphery of the top surface of the cap unit 2202. FIG. 30B depicts a side view of the marker 2200. The marker 2200 includes the spring loaded pin 2224 in the cap unit. The pin 2224 is angled upward away form the top surface of the cap unit 2202 and is configured to retract into and out of the cap unit 2204 by a spring unit (not shown). The marker 2200 also contains a plurality of barbs 2206 affixed to the lower portion of a cap unit 2202. In one embodiment, the barbs 2206 are arranged around the periphery of each of the openings 2204. The barbs 2206 include sharpened ends 2208 which face away from the center of the cap unit 2202. In one embodiment, the barbs 2206 are perpendicular to the lower surface of the cap unit 2202. In another embodiment, the barbs 2206 are angled away from the center of the cap unit 2202. In yet another embodiment, each of the barbs 2206 are angled in different directions relative to one another. In one embodiment, a hollow cylinder 2210 is attached to the lower portion of the cap unit 2202 around the opening 2204. In another embodiment, the barbs 2206 are attached to the hollow cylinder 2210. The distal end of the hollow cylinder 2210 may be blunt or sharp allowing it to be the mechanism which pierces the region 84 along with or instead of the barbs 2206.

FIG. 30C depicts a delivery unit 2212 for a marker 2200. The delivery unit 2212 includes a tube 2214 with an opening 2216. A plurality of markers 2200 are stacked in the tube 2214 such that the barbs 2206 of one marker 2200 are in contact with the cap unit 2202 of another marker 2200 in the tube 2214. A vertical transmission unit 2218 engages the opening 2204 in the cap unit 2202 and forces the marker 2200 closest to the opening 2216 to move towards a region 84. In another embodiment, a spacer (not shown) is used in between markers 2200.

In one embodiment, the vertical transmission unit 2218 is a blunt ended stylet that is configured to engage at least one opening in the cap unit 2202 such that the cap unit 2202 is supported by the blunt ended stylet during deployment. In another embodiment, the vertical transmission unit 2218 includes a plurality of blunt ended stylets which are configured to engage the plurality of openings 2204 on the top surface of the cap unit 2202. In yet another embodiment, the vertical transmission unit is a needle or a plurality of needles configured to engage at least one opening in the cap unit 2202.

In one embodiment, the vertical transmission unit 2218 engages the hollow cylinder 2210. In another embodiment, the vertical transmission unit 2218 is the hollow cylinder 2210. In another embodiment, the barbs 2206 are arranged on the lower surface of the cap unit 2202 such that the barbs 2206 are in contact with the surface of the vertical transmission unit 2218 when the vertical transmission unit engages the opening 2204. In another embodiment, the barbs 2206 are arranged around the plurality of openings 2204 such that the barbs 2206 are in contact with the portions of the vertical transmission unit 2218 passing through each of the plurality of openings 2204.

As the vertical transmission unit 2218 moves through the opening 2216, the sides of the vertical transmission unit 2218 press against the pin 2224 forcing the pin 2224 into the cap unit 2202. When a notch 2222 located on the side of the vertical transmission unit 2218 is positioned over the pin 2224, a spring unit (not shown) in the cap unit forces the pin 2224 into the notch 2222 preventing the vertical transmission unit 2218 from moving downward through the opening 2204 and positioning the end of the vertical transmission unit 2218 a predetermined distance below the barbs 2206.

As the vertical transmission unit 2218 continues to move downward, the vertical transmission unit 2218 forces the marker 2200 downward. In one embodiment, the vertical transmission unit 2218 is a needle that pierces the region 84 before the barbs 2206. In another embodiment, the vertical transmission unit 2218 is a needle which pierces the region 84 after the barbs 2006 have pierced the region 84. In another embodiment, the vertical transmission unit 2218 is a needle which pierces the region 84 at the same time the barbs pierce the region 84. In yet another embodiment, the vertical transmission unit 2218 is a blunt stylet that does not pierce the region 84. In another embodiment, the vertical transmission unit 2218 includes a first transmission portion which moves the vertical transmission unit 2218 downward and a second transmission portion which separately moves the marker 2200 downward.

Once the barbs 2206 pierce the region 84, the vertical transmission unit 2218 is retracted back through the opening 2204 in the cap unit 2202. As the vertical transmission unit 2218 moves backward, the pin 2224 disengages the notch 2222 and the sides of the vertical transmission unit 2218 force the pin 2224 into the cap unit 2202. In another embodiment, the pin 2224 is located on the vertical transmission unit 2218 and the notch 2222 is located in the cap unit. Consistent with this embodiment, the pin 2224 is coupled to an engagement unit (not shown) that allows a user of the vertical transmission unit 2218 to retract and eject the pin 2224 from the vertical transmission unit 2218. FIG. 30E depicts the marker 2200 engaged with the region 84 after the vertical transmission unit 2218 is removed. In another embodiment, more than one pin 2224 is employed. In another embodiment, a different engagement mechanism is employed (i.e. pincers). In yet another embodiment, no distal engagement mechanism is necessary as the vertical transmission unit 2218 is designed to recoil automatically into the delivery catheter once the marker 2200 has pierced and/or has been deployed into the region 84 or because the vertical transmission unit has been deployed into the region 84 with or alongside the marker 2200.

In one embodiment, the vertical transmission unit 2218 engages the opening 2204 and passes through the hollow cylinder 2210 which moves the hollow cylinder 2210 towards the region 84 until the hollow cylinder 2210 pierces the region 84. In one embodiment, the cap unit 2202 is color coded to indicate the location of the marker 2200. In another embodiment, the cap unit 2202 includes an RFID tag to indicate the location of the marker 2200. In another embodiment, the cap unit 2202 includes a letter, number or symbol on the surface of the cap unit 2202.

In all of the embodiments described herein, one or a plurality of markers are inserted around, adjacent to, or on a region of interest to mark the location to assist with a current examination or for another examination at a later time. In one embodiment, groups of markers are associated with a specific region using by coloring a portion of the marker with a unique color or with marking the marker with a number. In another embodiment, RFID tags are inserted into the markers and are used to associate a group of markers with a specific region. In yet another embodiment, the markers may be bent to indicate a number. As an illustrative example, ten markers may be arranged around a region. Each marker may be bent upward to indicate a “1” or “On” state and other markers may be bent downwards to indicate a “0” or “Off” state. A user or device may then read the 10 markers to generate a binary number used to identify the region. In another embodiment, the top portions of the markers may be formed in to a geometric shape.

In addition, each of the markers disclosed is deployed and/or implanted into the human body. As a result, all of the components of the markers are made from materials which are not toxic to the human body including, but not limited to, stainless steel, titanium, plastic, polymers, ceramic, rubber, nitinol, biodegradable or any other non toxic material, or combination of materials, capable of performing the required function for the marker.

These markers may be used in numerous applications including, but not limited to, marking polyps, marking lesion perimeters, marking of pre-op cancer, pre-operative diverticulosis extent marking, AVM's to identify suite of future bleeding, marking the extent of ulcerative colitis, marking a submucosal lesion for future EUS, functioning as an anchor, or any other suitable use.

The disclosed embodiments are illustrative of the various ways in which the present invention may be practiced. Other embodiments may be implemented by those skilled in the art without deporting from the spirit and scope of the present invention.

1. A marker for marking the location of an internal area in the human body comprising:
a cap unit; and
at least one attachment unit affixed to the cap unit, the at least one attachment unit capable of piercing a surface of a region in response to a vertical force applied to the cap unit.

2. The marker of claim 1, wherein the cap unit is at least one of: color coded, a radio frequency identification unit, or dissolves over time.

3. The marker of claim 1, wherein characters are at least one of: indented, notched, etched, printed, written with laser into a top portion of the cap unit.

4. The marker of claim 1, wherein the at least one attachment unit comprises at least one of micro-bristles, barbs, an adhesive layer, or pins.

5. The marker of claim 1, wherein the at least one attachment unit is capable of piercing the surface of the region in response to a rotational force applied to the cap unit.

6. The marker of claim 1, wherein the at least one attachment unit is capable of piercing the surface of the region in response to a horizontal force applied to the cap unit.

7. The marker of claim 1, wherein the at least one attachment unit is capable of piercing the surface of the region in response to a diagonal force applied to the cap unit.

8. The marker of claim 1, further comprising:
a delivery unit comprising:
a tube holding a plurality of markers; and
a vertical transmission unit configured to apply a vertical force to a cap unit of a marker to cause the marker to pierce the surface of the region.

9. A marker for marking the location of an internal area in the human body comprising:
a cap unit; and
a screw unit affixed to the cap unit, the screw unit capable of piercing a surface of a region in response to a rotational force applied to the cap unit.

10. The marker of claim 9, wherein the cap unit is at least one of: color coded, a radio frequency identification unit, or dissolves over time.

11. The marker of claim 9, wherein characters are at least one of: indented, notched, etched, printed, written with laser into a top portion of the cap unit.

12. The marker of claim 9, further comprising:
a delivery unit comprising:
a tube holding a plurality of markers; and
a screw mechanism configured to apply a rotational force to a cap unit of a marker to cause the marker to pierce the surface of the region.

13. The marker of claim 9, wherein the cap unit comprises a plurality of grooves to engage at least one gear unit causing a rotational force to be applied to the cap unit.

14. A marker for marking the location of an internal area in the human body comprising:
a cap unit; and
a plurality of attachment units affixed to sides of an opening in a lower portion of the cap unit, the plurality of attachment units capable of piercing a surface of a region in response to a suction force applied to the cap unit.

15. The marker of claim 14, wherein the cap unit is at least one of: color coded, a radio frequency identification unit, or dissolves over time.

16. The marker of claim 14, wherein characters are at least one of: indented, notched, etched, printed, written with laser into a top portion of the cap unit.

17. The marker of claim 14, wherein the plurality of attachment units comprises at least one of micro-bristles, barbs, or pins.

18. The marker of claim 14, further comprising:
a delivery unit comprising:
a suction tube holding a plurality of markers; wherein the suction tube applies suction causing a portion of the region to extend upwards into a cavity of the marker causing the marker to pierce the surface of the region.

19. A method for administering a marker to an internal area in a human body, comprising:
loading a delivery unit with at least one marker;
placing the delivery unit at a region to be marked; and
activating a control device of the delivery unit to deploy a marker of the at least one marker, causing the marker to pierce a surface of the region.

20. The method of claim 19, wherein loading a delivery unit with at least one marker comprises:
loading the delivery unit with a packet containing a plurality of markers.

21. The method of claim 19, wherein the control device is at least one of: a gun-type control, a knob, or a lever to control deployment of markers.

22. The method of claim 19, wherein activating a control device of the delivery unit to deploy a marker of the at least one marker comprises:
activating a vertical transmission unit that forces a marker closest to an opening of the delivery unit to pierce the surface of the region.

23. The method of claim 19, wherein the vertical transmission unit is a blunt ended stylet.

24. A mirror device including:
an extension unit;
a holding unit on the extension unit made from a material having memory capabilities;
a flexible reflective material secured to the holding unit.

25. The mirror device of claim 24, wherein the holding unit and the flexible reflective material form a convex mirror.

26. The mirror device of claim 24, wherein the holding unit and the flexible reflective material form a concave mirror.

27. The mirror device of claim 24, wherein the flexible reflective material is opposing sides of the holding unit.

28. The mirror device of claim 27, wherein the holding unit and the flexible reflective material form both a concave and convex mirror.
